SEQUENCE LISTING (17 sequences)

SEQ ID NO: 1 - DNA, 882 bp, Staphylococcus aureus
gcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaa

SEQ ID NO: 2 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 3 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGDIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 4 - DNA, 37 bp, Artificial
  synthetic
aactttactcccttcctccccgccccccccccccccc

SEQ ID NO: 5 - DNA, 53 bp, Artificial
  synthetic
gagcaaaggtattaactttactccccccccccccccccccccccccccccccc

SEQ ID NO: 6 - DNA, 53 bp, Artificial
  synthetic
tgctgcggttattaaccacaacacccccccccccccccccccccccccccccc

SEQ ID NO: 7 - DNA, 53 bp, Artificial
  synthetic
tacattgtactcattccaattaacccccccccccccccccccccccccccccc

SEQ ID NO: 8 - DNA, 53 bp, Artificial
  synthetic
atgtgcacagttacttacacatacccccccccccccccccccccccccccccc

SEQ ID NO: 9 - DNA, 22 bp, Artificial
  synthetic
aacgtcaatgagcaaaggtatt

SEQ ID NO: 10 - DNA, 21 bp, Artificial
  synthetic
ctgaaagtactttacaacccg

SEQ ID NO: 11 - DNA, 37 bp, Artificial
  synthetic
aactttactcccttcctcccggccccccccccccccc

SEQ ID NO: 12 - DNA, 37 bp, Artificial
  synthetic
atggcaagaggcccgaaggtccccccccccccccccc

SEQ ID NO: 13 - DNA, 37 bp, Artificial; identical to SEQ ID NO: 12
  synthetic

SEQ ID NO: 14 - DNA, 37 bp, Artificial
  synthetic
tcagaccagctagggatcgtcgccccccccccccccc

SEQ ID NO: 15 - DNA, 37 bp, Artificial; identical to SEQ ID NO: 4
  synthetic

SEQ ID NO: 16 - DNA, 37 bp, Artificial
  synthetic
tcagtcttcgtccagggggccgccccccccccccccc

SEQ ID NO: 17 - DNA, 37 bp, Artificial
  synthetic
gccatgcagcacctgtctcacgccccccccccccccc

(19) United States
(12) Patent Application Publication
(10) Pub. No.: US 2018/0155768 A1
(43) Pub. Date: Jun. 7, 2018

(54) NUCLEIC ACID DETECTION

(71) Applicant: NANOPORE DIAGNOSTICS, LLC, Saint Louis, MO (US)

(72) Inventors: Thomas L. COHEN, Saint Louis, MO (US); Somes K DAS, Maryland Heights, MO (US); Julie REGELIN, Maryland Heights, MO (US)

(73) Assignee: NANOPORE DIAGNOSTICS, LLC, Saint Louis, MO (US)

(21) Appl. No.: 15/578,821

(22) PCT Filed: Jun. 1, 2016
(86) PCT No.: PCT/US2016/035256

§ 371 Date: Dec. 1, 2017

Related U.S. Application Data
(60) Provisional application No. 62/169,672, filed on Jun. 2, 2015.
(60) Provisional application No. 62/253,170, filed on Nov. 10, 2015.

(51) Int. Cl. C12Q 1/6825

(52) U.S. Cl. CPC C12Q 1/6825; C12Q 2565/631; C12Q 2521/327

(57) ABSTRACT

Systems, devices, and methods of detecting nucleic acids may include a nanopore system or use of a nanopore system. The method of detecting a target nucleic acid may include combining a sample with at least one with at least one probe molecule having a sequence fully complementary or partially complementary to the target nucleic acid, such that the probe molecule hybridizes to the target nucleic acid, and adding one of more enzymes before or after combining the sample with the probe molecule(s). The sample may be added to a chamber of a nanopore system and a voltage applied to generate a current time series, wherein a signature current pattern of the nanopore system indicates the presence of the target nucleic acid in the sample.

This application claims the benefit of priority to U.S. Provisional Application No. 62/169,672, filed on Jun. 2, 2015; and U.S. Provisional Application No. 62/253,170, filed on Nov. 10, 2015, each of which is incorporated by reference herein in its entirety.

### TECHNICAL FIELD

The present disclosure generally relates to the detection of nucleic acids and oligonucleotides with a nanopore-based system.

### BACKGROUND

One of the main challenges in healthcare today is providing patients with prompt, accurate, and cost-effective diagnostic information. Many diseases have a complex etiology that can be particularly difficult to identify at the earliest stages of disease, when treatment is typically most effective.

Certain biomarkers can be indicative of a disease state. For example, micro RNAs (miRNAs) are short, non-coding RNA molecules or fragments of RNA molecules that regulate gene expression at the post-transcriptional level. Depending on the degree of homology to their target sequences, miRNA binding induces translational repression or cleavage of mRNAs. As gene regulators, miRNAs are understood to play a role in development, cell differentiation, and regulation of cell cycle, apoptosis and signaling pathways. Aberrant expression of miRNAs may provide an indication of a disease state, including genetic diseases as well as pathogen-caused illnesses. Many other types of nucleic acid biomarkers also have been found useful for medical diagnosis, including larger nucleic acids such as genomic DNA (gDNA), messenger RNA (mRNA), and for microbial diagnostics, ribosomal RNA (rRNA).

A pathogen-related health condition that is often difficult to diagnose is sepsis, a life-threatening response by the body to a severe infection. Widespread inflammation induced by the body to fight the infection can lead to low blood pressure and organ dysfunction. Sepsis can kill quickly, with mortality rates increasing about 8% every hour before treatment begins. One of the main challenges in treating sepsis and other conditions caused by infectious agents is rapidly identifying the optimal anti-microbial treatment for a particular patient. Although many different microbes can cause sepsis (e.g., bacteria, fungi, viruses), most cases are bacterial.

Detection and quantification can present significant analytical hurdles, not to mention the time involved in testing and analysis to reach a diagnosis. Table 1 below lists some examples of techniques used for diagnostic analysis of blood-borne infections.

TABLE 1

| | | Time-to-diagnosis | | | |
|---|---|---|---|---|---|
| Method | Example | (blood sample) | | Point-of-care? | Culture? |
| Culture analysis | Vitek (Biomerieux) | 2-5 | days | No | Yes |
| Lateral-flow assay | BinaxNow (Alere) | 1-2 | days | Yes | Yes |
| Mass spectrometry | Vitek MS (Biomerieux) | 1-2 | days | No | Yes |
| Nanoparticle detection | Nanosphere Verigene | 1-2 | days | No | Yes |
| DNA microarray | Mobidiag Prove-it | 1-2 | days | No | Yes |
| PNA FISH | AdvanDx PNA Fish | 1-2 | days | No | Yes |
| qPCR-based platforms | FilmArray (BioFire) | 16 | hours | No | Yes |
| Combination platforms | Iridica (Abbott) | 8 | hours | No | No |
| Magnetic resonance | T2MR (T2-Biosystems) | 5 | hours | No | No |

In the case of a blood-borne pathogen, culture analysis can take several days to a week to identify the microbe present, during which immediate broad-spectrum antibiotics may be prescribed, combined with measures to treat sepsis symptoms. Doctors then move from broad-spectrum antibiotics to more specific treatments when test results return days later. Yet, aggressive broad-spectrum treatments can produce side effects and potentially lead to antibiotic resistance. In addition, broad-spectrum antibiotics are not effective against all microbes and other inflammatory conditions associated with symptoms similar to sepsis.

Additional technologies used for nucleic acid detection include reverse transcription real-time polymerase chain reaction (RT-qPCR) and microarrays. These techniques often require enzymatic amplification and produce only qualitative or semi-quantitative results. Further, such techniques still require a culture procedure upstream of analysis due to sensitivity limits and are not well-suited to point-of-care analysis. And detection of large nucleic acids by these or other available techniques can involve long processing times, which may be unsatisfactory in a clinical setting.

### SUMMARY

The present disclosure includes a method of detecting a target nucleic acid in a sample, comprising combining the sample with at least one probe molecule having a sequence fully complementary or partially complementary to the target nucleic acid, the target nucleic acid being single-stranded, such that the probe molecule hybridizes to the target nucleic acid; combining the sample with one or more enzymes to produce a probe/target complex; applying a voltage across a nanopore system while the probe/target complex is on a first side of a partition of the nanopore system, the partition including a nanopore defining a channel; and analyzing an electrical current of the nanopore system over time, wherein a presence of the target nucleic acid in the sample is indicated by a signature current pattern. The signature current pattern may comprise a level or a series of levels having magnitudes of current and durations respectively different from magnitudes of current and/or durations of levels of each of an electrical current that occurs with the sample in absence of the at least one probe molecule and an electrical current that occurs with the at least one probe molecule in absence of the target nucleic acid. In some aspects, the sample may comprise a parent nucleic acid that includes the sequence of the target nucleic acid, such that the target of the probe/target complex is a fragment of the parent nucleic acid. The parent nucleic acid may be a single-stranded nucleic acid or a double-stranded nucleic acid (which, in some aspects, may be denatured or otherwise processed to separate the double strand into single strands). Combining the sample with the one or more enzymes may cleave the parent nucleic acid to isolate the probe/target complex from a remainder of the parent nucleic acid. In at least one example, the target nucleic acid (or both the parent nucleic acid and the target nucleic acid) may comprise RNA. In some aspects, the sample may comprise non-target single-stranded nucleic acids, and the one or more enzymes may digest the non-target single-stranded nucleic acids. Accordingly, such treatment may reduce or eliminate signals associated with non-target nucleic acids and/or other species of the sample other than the target nucleic acid (background signals).

According to some aspects, the one or more enzymes comprises RNase A, RNase 1, RNase 1f, RNase P, RNase PhyM, RNase T1, RNase T2, RNase U2, or a mixture thereof, such as, e.g., RNase 1f or a mixture of RNase A and RNase T1. The target nucleic acid may comprise from 15 to 25 nucleotides in length, such as from 18 to 25 nucleotides, or from 19 to 24 nucleotides, e.g., 19, 20, 21, 22, 23, or 24 nucleotides in length. The parent nucleic acid (which may include the sequence of the target nucleic acid) may comprise more than 30 nucleotides in length (or more than 30 base pair, bp), more than 40 nucleotides, or more than 50 nucleotides in length. In at least some examples, the parent nucleic acid may comprise 100 or more nucleotides in length, e.g., from 100 to 2000 nucleotides (or from 100 to 2000 bp), such as from 200 to 1800 nucleotides, from 300 to 1700 nucleotides, or from 400 to 1700 nucleotides, or from 500 to 1600 nucleotides.

The method may comprise combining the sample with more than one probe molecule and/or more than one enzyme or enzymatic mixture, in the same or different steps of the method. For example, in the exemplary method mentioned above, the at least one probe molecule may be a third probe molecule (e.g., a third DNA probe molecule), wherein the method further comprises combining the sample with a first probe molecule having a sequence complementary to a sequence of the parent nucleic acid flanking a 3′ end of the target nucleic acid and a second probe molecule having a sequence complementary to a sequence of the parent nucleic acid flanking a 5′ end of the target nucleic acid, before combining the sample with the third probe molecule. The first, second, and third probe molecules may include DNA sequences, e.g., the first probe molecule being a first DNA probe molecule, the second probe molecule being a second probe DNA molecule, and the third probe molecule being a third probe DNA molecule. Thus, for example, the parent nucleic acid and the target nucleic acid may comprise RNA nucleotides.

In some aspects, the method may further comprise combining the sample with one or more enzymes (e.g., an enzymatic mixture) after combining the sample with the first and second probe molecules (e.g., the first and second DNA probe molecules) and before combining the sample with the third probe molecule (e.g., the third DNA probe molecule). The enzyme(s) may cleave the parent nucleic acid at the 3′ end and the 5′ end of the target nucleic acid to release the target nucleic acid from the parent nucleic acid. In some aspects, for example, the enzyme(s) may comprise RNase H.

Detection of the target nucleic acid may comprise analysis of a signature current pattern of the nanopore system. The signature current pattern may comprise, for example, two, three, four, or five or more consecutive levels of electrical current. Each level may have a magnitude of current different from the adjacent levels. In some examples, each level may have a magnitude of current different from each of the other levels of signature current pattern. Further, in some aspects, each level of the signature current pattern may have a duration different from the duration of at least one of the other levels, or may have a duration different from each of the other levels.

The target/probe complex may enter the cis opening or the trans opening of the nanopore to product the signature current pattern. In some aspects, the signature current pattern may correspond to: (a) trapping the target/probe complex in a trans opening of the nanopore; or (b) detaching the target nucleic acid from the at least one probe molecule of the target/probe complex and translocating at least one of the probe molecule or the target nucleic acid completely through the nanopore. In some examples, the target/probe complex may enter the cis opening of the nanopore, followed by detaching the target nucleic acid from the at least one probe molecule of the target/probe complex and translocating at least one of the probe molecule or the target nucleic acid completely through the nanopore. In other examples, the target/probe complex may enter the trans opening of the nanopore, followed by detaching the target nucleic acid from the at least one probe molecule of the target/probe complex and translocating at least one of the probe molecule or the target nucleic acid completely through the nanopore, from a cis opening to a trans opening of the nanopore, or from the trans opening to the cis opening of the nanopore. In at least one example, the signature current pattern may comprise three consecutive levels of electrical current, each level having a magnitude of current different from the other two levels of the three consecutive levels.

When the sample comprises other, non-target nucleic acids, the signature current pattern may distinguish the target nucleic acid from the non-target nucleic acids. Additionally or alternatively, the sample may comprise two or more target nucleic acids, each target nucleic acid having a signature current pattern different from the signature current patterns of the other target nucleic acids, e.g., to allow for distinguishing among the different targets. In some aspects, the signature current patterns may be analyzed to determine the concentration of each target nucleic acid in the sample. The various target nucleic acids may have similar sequences, e.g., differing by only 1, 2, 3, or 4 nucleotides in some examples. Thus, for example, the sample may comprise a first target nucleic acid and a second target nucleic acid, wherein a sequence of the second target nucleic acid differs from the sequence of the first nucleic acid by 1 or 2 nucleotides, wherein the signature current pattern (e.g., corresponding to a target/probe complex formed from the first nucleic acid and the at least one probe molecule) distinguishes the presence of the first nucleic acid in the sample from the presence of the second nucleic acid in the sample.

The nanopore system may comprise a first chamber that includes the first side of the partition and a second chamber that includes a second side of the partition. To provide the voltage, the nanopore system may comprise a negative electrode and a positive electrode. In some aspects, the first chamber may be in contact with the negative electrode, and the second chamber may be in contact with the positive electrode. In other aspects, the second chamber may be in contact with the negative electrode, and the first chamber may be in contact with the positive electrode.

The nanopore may comprise a biological nanopore or a synthetic nanopore. In some examples, the partition may include a plurality of nanopores chosen from biological nanopores, synthetic nanopores, or a combination thereof. In some aspects, the channel of each nanopore may have a minimum cross-sectional size ranging from about 1.2 nm to about 1.8 nm. For channels with a circular cross-sectional shape, for example, the minimum diameter of the channel may range from about 1.2 nm to about 1.8 nm.

With respect to biological nanopores, in some examples the nanopore may comprise Staphylococcus aureus α-hemolysin (or a variant thereof) or Mycobacterium smegmatis porn A (or a variant thereof), or Escherichia coli CsgG (or a variant thereof). With respect to synthetic nanopores, in some example, the nanopore may comprise comprises silicon, silicon dioxide (SiO₂), silicon nitride (Si₃N₄), molybdenum disulfide (MoS₂), aluminum oxide (Al₂O₃), boron nitride (BN), graphene, or a combination thereof. The channel may be defined by a surface of the nanopore that includes a plurality of molecules or chemical functional groups facing radially inward, e.g., such that the channel is functionalized. According to some aspects of the present disclosure, at least a portion of the surface of the nanopore defining the channel may comprise a plurality of DNA hairpin loops, a plurality of polypeptide molecules, or a mixture thereof. Further, for example, the surface of the nanopore may comprise a plurality of molecules having a sequence at least partially complementary to the sequence of the target nucleic acid, wherein the plurality of molecules may or may not include DNA hairpin loops or polypeptide molecules.

The target nucleic acid may be a biomarker. For example, the target nucleic acid may be a biomarker of a genetic disease, an environmental disease, an organism genotype, a pathogen, or a resistance to an antibiotic. In some aspects, the target nucleic acid may be a biomarker of, or associated with, two or more of a genetic disease, an environmental disease, an organism genotype, a pathogen, or a resistance to an antiobiotic. According to some aspects, the target nucleic acid may comprise a fragment of whole RNA, such as a fragment of microbial rRNA, e.g., a fragment of bacterial rRNA, or the target nucleic acid may comprise a microRNA. In at least one example, the target nucleic acid may be a biomarker of a bacterial infection.

The method may further comprise quantifying an amount of the target nucleic acid and/an amount of the parent nucleic acid in the sample. Thus, for example, the concentration of the target nucleic acid in a sample may be quantified to obtain diagnostic information about a disease or other health condition. In some examples, the target nucleic acid and/or the parent nucleic acid may be detected and quantified within about 2 hours, e.g. less than 90 minutes, less than 1 hour, or less than 30 minutes. The sample may comprise blood, may be obtained from blood, may comprise a liquid other than blood (including, e.g., biological liquids such as urine, mucus, bile, lymph, sweat, saliva, gastric acid, or peritoneal fluid, among other examples of biological fluids), or may comprise a liquid derived from tissue.

The present disclosure also includes a method of detecting a target nucleic acid in a sample, the method comprising combining the sample with a first probe molecule and a second probe molecule, wherein the sample comprises a parent nucleic acid that includes a sequence of the target nucleic acid; the first probe molecule has a sequence complementary to a sequence of the parent nucleic acid flanking a 3′ end of the target nucleic acid; and the second probe molecule has a sequence complementary to a sequence of the parent nucleic acid flanking a 5′ end of the target nucleic acid; the method further comprising adding at least one first enzyme to the sample; combining the sample with a third probe molecule having a sequence fully complementary or partially complementary to the sequence of the target nucleic acid, such that the third probe molecule hybridizes to the target nucleic acid; adding at least one second enzyme to the sample to produce a target/probe complex; and detecting the target/probe complex with a nanopore system. In some examples, the at least one first enzyme may comprise RNase H, and the at least one second enzyme may be chosen from RNase A, RNase 1, RNase 1f, RNase P, RNase PhyM, RNase T1, RNase T2, RNase U2, or a mixture thereof (e.g., RNase 1f or a mixture of RNase A and RNase T1). The nanopore system may be any of the exemplary systems disclosed herein. In some examples, detecting the target/probe complex may comprise: applying a voltage across a nanopore system while the probe/target complex is on a first side of a partition of the nanopore system, the partition including a nanopore defining a channel; and analyzing an electrical current of the nanopore system over time, wherein a presence of the target nucleic acid in the sample is indicated by a signature current pattern. The signature current pattern may comprise level or a series of levels having magnitudes of current and durations respectively different from amplitudes and durations of levels of each of an electrical current that occurs with the sample in absence of the third probe molecule and an electrical current that occurs with the third probe molecule in absence of the target nucleic acid.

The present disclosure further includes nanopore systems and devices comprising nanopore systems suitable for performing the methods described above and elsewhere herein. For example, the device may comprise a cartridge that includes one or more nanopore systems as disclosed herein. In some examples, the device may comprise two or more cartridges, each including one or more nanopore systems as disclosed herein. The cartridge(s) may be insertable into a slot of the device, such that the cartridge(s) are removable, e.g., allowing for new cartridges to be inserted for each assay. Each cartridge may include a plurality of wells, and the nanopore system(s) may be included in at least one of the wells. According to some aspects of the present disclosure, each cartridge may include a plurality of nanopore systems each disposed in a different well of the cartridge, and each nanopore system designed to detect a different target nucleic acid. Thus, for example, the device may be configured to detect at least 10 different target nucleic acids. In some aspects, each nucleic acid may be a biomarker, such that the device may provide diagnostic information about the presence of a disease or other health condition, or the likelihood of contracting a disease or other health condition. In at least one example, the device may be portable. In at least one example, the device may be a point-of-treatment device. In some aspects, the device may be configured to detect and/or quantify the target nucleic acid(s) in less than about 2 hours, less than about 90 minutes, less than about 1 hour, or less than about 90 minutes.

### BRIEF DESCRIPTION OF THE DRAWINGS

The accompanying drawings, which are incorporated in and constitute a part of this specification, illustrate various examples and together with the description, serve to explain the principles of the present disclosure. Any features of an embodiment or example described herein (e.g., system, device, method, etc.) may be combined with any other embodiment or example, and are encompassed by the present disclosure.

FIG. 1 is a schematic of an exemplary nanopore system according to some aspects of the present disclosure.

FIG. 2A illustrates an exemplary time-series of current measured for a nanopore system according to some aspects of the present disclosure, including a magnified portion of the time series showing an exemplary signature pattern.

FIGS. 2B and 2C show additional exemplary signature patterns for nanopore systems in accordance with the present disclosure.

FIGS. 3A-3F illustrate examples of nanopores according to some aspects of the present disclosure.

FIG. 4 is a schematic of a solid-state nanopore channel (ssNPC) according to some aspects of the present disclosure.

FIGS. 5A-5D are schematics of additional examples of ssNPC) in accordance with some aspects of the present disclosure.

FIG. 6 shows examples of different signature patterns corresponding to different types of probe molecules in accordance with some aspects of the present disclosure.

FIGS. 7A and 7B are schematics of probe molecules comprising multiple tags, in accordance with aspects of the present disclosure.

FIGS. 8, 9, and 10 are schematics of exemplary assays, according to some aspects of the present disclosure.

FIG. 11 shows an exemplary device comprising one or more nanopore systems in accordance with the present disclosure.

FIG. 12 shows results of gel electrophoresis for E. coli rRNA, discussed in Example 1.

FIG. 13 compares assay results of different microbial species, discussed in Example 2.

FIG. 14 shows results of gel electrophoresis for a 90 bp RNA, discussed in Example 3.

FIG. 15 shows results of gel electrophoresis for E. coli rRNA, discussed in Example 4.

FIGS. 16A and 16B show graphs of current blockage duration vs. magnitude of current blockage, discussed in Example 5.

FIGS. 17A and 17B show graphs of current blockage duration vs. magnitude of current blockage, discussed in Example 6.

### DETAILED DESCRIPTION

Embodiments of the present disclosure include systems and methods for detecting nucleic acids and fragments thereof, including oligonucleotides, which may be indicative of a disease or other health condition. Aspects of the present disclosure may assist in and/or offer certain advantages in point-of-care diagnosis, in lab-based diagnostics, for research and in other non-clinical settings, and/or in non-medical applications. For example, some aspects of the present disclosure may be useful in clinical testing, e.g., to allow a healthcare provider to administer a more individualized or targeted treatment of a patient during the patient's visit or shortly following an examination of the patient. Further, for example, some systems herein may be useful as a research tool. Non-medical applications of aspects of the present disclosure include, but are not limited to, food safety, sterility, and/or agricultural testing.

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass ±5% of a specified amount or value.

The present disclosure may include any of the devices, systems, and/or methods, or any features thereof, disclosed in U.S. Application Publication No. 2013/0220809 and/or U.S. Application Publication No. 2014/0309129, each of which is incorporated by reference herein.

Systems according to the present disclosure may comprise one or more nanopores comprising molecular-scale pore structures. Each nanopore may define a channel having a cross-sectional size that selectively limits the passage of chemical or biochemical species therethrough. In some aspects, for example, the nanopore(s) may have a minimum cross-sectional size that allows the passage of single-stranded nucleic acids through the channel but prevents passage of double-stranded nucleic acids. The nanopore(s) may be incorporated into an insulating membrane or partition between two chambers each in contact with an electrode, such that a voltage applied across the membrane may generate an electrical current through the channel(s) of the nanopore(s). Individual chemical or biochemical species of interest (targets) passing through each channel may block the current in a characteristic pattern, which may be used for detection, identification, and/or quantification of the target(s) of interest. The nanopore system therefore may serve as a sensor useful for detecting single target molecules by monitoring blocks in current flow.

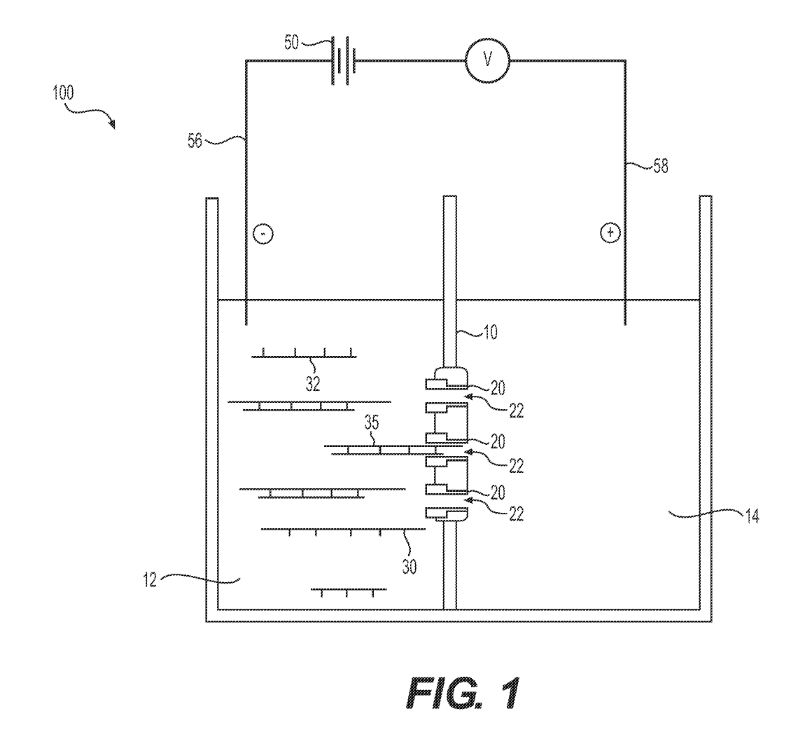

FIG. 1 shows an exemplary system 100 according to some aspects of the present disclosure. The system may include a partition 10 between two chambers 12, 14, at least one nanopore 20 incorporated into the partition 10 (three nanopores 20 as shown in this example), at least one probe molecule 30 in one of the chambers 12, a power source 50, and a pair of electrodes 56, 58 operably coupled to the power source 50. Each nanopore 20 may define a channel 22, such that a voltage applied to the partition 10 may generate current through the channels 22.

In the nanopore systems herein, the side of the nanopore(s) facing the negative electrode is referred to herein as the cis side (which includes the cis opening of the nanopore), and the opposite side facing the positive electrode is referred to as the trans side (which includes the trans opening of the nanopore). Further, the chamber in contact with the negative electrode is referred to as the cis chamber, and the chamber in contact with the positive electrode is referred to as the trans chamber. Thus, in the example shown in FIG. 1, chamber 12 may be referred to as the cis chamber, and chamber 14 may be referred to as the trans chamber. In some examples, one opening of the nanopore 22 may be wider than the other opening, e.g., the cis opening may be wider than the trans opening, or vice versa, as illustrated here.

The probe molecule 30 may comprise a nucleic acid sequence fully or partially complementary to the sequence of a target oligonucleotide of interest 32, e.g., such that the probe molecule 30 and the target 32 may hybridize to form a double-stranded target/probe oligonucleotide complex 35. A sample to be analyzed may be added to the chamber 12 that includes the probe molecule 30, such that targets 32 in the sample may hybridize to respective probe molecules 30 to produce target/probe complexes 35.

The power source 50 may provide a pre-determined voltage, e.g., as a driving force for target/probe complexes 35 to enter the channels 22 of the nanopores 20, e.g., to induce separation of the target 32 from the probe molecule 30 (e.g., unzipping of the double-stranded oligonucleotide complex 35) due at least in part to the size constraints of the nanopore channel 22. This separation of the complex 35 may be followed by translocation of the probe molecule 30 and/or the target 32 through the channel 22. In some aspects, the target/probe complex 35 may be temporarily trapped in the channel 22, and may not separate to allow the individual probe molecule 30 and/or target 32 to translocate through the channel 22 but instead release back into the original chamber 12. These separation, translocation, and trapping events may produce a series of characteristic blockages of current through the nanopore channels 22, which may be analyzed to detect, identify, and/or quantify the targets 32 present in the sample. Such a series of current blockages is referred to herein as a signature pattern.

Signature Patterns

Signature patterns may be used to distinguish target/probe complexes from other components in a sample, such as free (unbound) probe molecules, free (unbound) target nucleic acids, non-target single- and double-stranded nucleic acids, and molecules other than nucleic acids or probe molecules (e.g., small peptides and other polymers). These other events may be termed background events. A signature pattern may be characterized by one or more of the following: the number of consecutive blockages within a series (e.g., the number of “levels” of a series); the magnitude of current during each level (e.g., as compared to an open, unblocked nanopore); the duration of each level; and/or the magnitude of current of a given level relative to one or more other levels of the series.

The number of levels of a series (not including the current of an open, unblocked pore) may range from 1 to 50 or more, depending on features of the target/probe complex and the nanopore. In some examples, the signature pattern may include 1, 2, 3, 4, 5, 6, 7, 8, 9, 10, 11, 12, 13, 14, or 15 levels, each level corresponding to a different magnitude of current as compared to the preceding or following level. Each level may have the same or different duration as compared to any other level of the signature pattern. In some aspects, signature patterns may be used to distinguish between two different targets within the same sample, as discussed below.

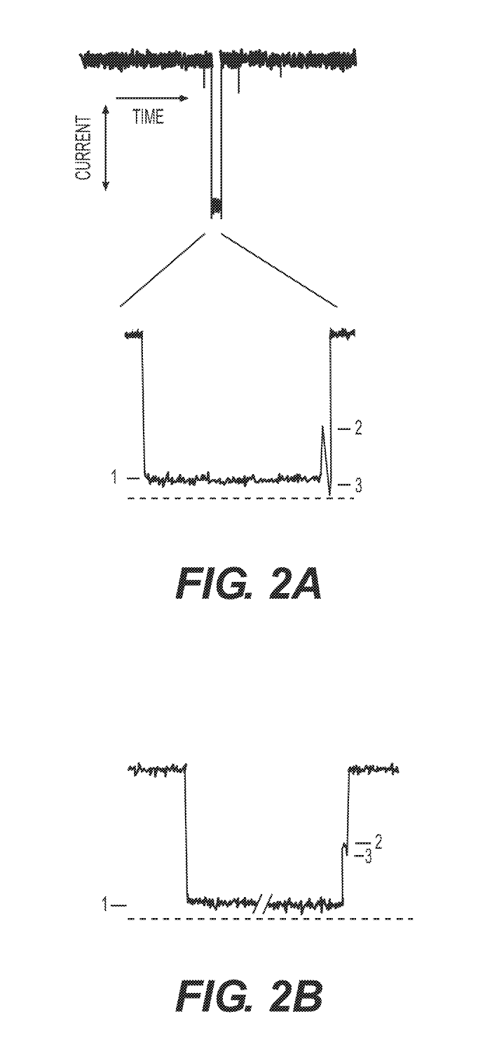

FIG. 2A shows an exemplary current time series including a 3-level signature pattern, also shown magnified. As shown, the magnitude of current decreases to level 1 (e.g., partial or total blockage of the nanopore channel), increases briefly to level 2 (e.g., partial opening of the nanopore to allow more current to pass), decreases to level 3, and then returns to the original unblocked level of current. FIGS. 2B and 2C show additional exemplary 3-level signature patterns, showing a similar series of levels but with variations in the durations and magnitudes of current of the levels.

Without intending to be bound by theory, it is believed that this type of 3-level pattern is consistent with trapping of a target/probe complex in the wider opening of a nanopore (e.g., cis opening in FIG. 1) (level 1), separation of the target from the probe molecule induced by the voltage and size constraints of the nanopore channel, followed by translocation of the probe molecule and temporary trapping of the target in the nanopore cavity (level 2), and translocation of the target through the channel (level 3).

Other exemplary signature patterns may have 2 levels. For example, level 1 may correspond to complete or nearly complete blockage of the nanopore channel by the target/probe complex, followed by separation of the target from the probe molecule and translocation of the probe molecule; and level 2 may correspond to temporary trapping of the target in the nanopore cavity, followed by release of the target from the nanopore cavity into the cis chamber, without translocation. The physical and/or chemical properties of the target and the probe molecule may affect the magnitude and/or duration characterizing each level.

In some examples, the signature pattern for a target/probe complex may range from about 5 ms to about 10000 ms in duration, and from about 140 pA to about 160 pA in current magnitude when 150 mV is applied in a recording solution of 1M KCl. Current blockages due to the probe molecule alone under these same conditions may range from about 1 μs to about 1000 μs in duration, and from about 80 pA to about 150 pA in current magnitude, and do not exhibit the unzipping signatures depicted in FIGS. 2A-2C, e.g., providing multiple boundaries to collect signal for each type of event. In some aspects of the present disclosure, the sample may be treated to remove or reduce non-target species, as discussed below, to assist in detection of one or more targets of interest. Measuring the frequency of the signature patterns observed may allow for quantification of the target nucleic acid(s).

Samples

A sample for testing as described herein may be obtained or derived from any subject of interest, including mammalian subjects, both human and non-human, as well as other biological materials. In some aspects, for example, the sample may be obtained from a human subject, e.g., a patient. Other mammalian subjects for which samples may be analyzed according to the systems and methods herein include, but are not limited to, non-human primates, cats, dogs, cattle, sheep, pigs, horses, chickens, and other domesticated or wild animals. The samples may be non-clinical. For example, samples may comprise, or be derived from, materials suspected of biological contamination, including, but not limited to, food products, drugs (including pharmaceuticals, biologics, veterinary drugs, and over-the-counter therapeutics), water supplies (e.g., municipal water sources), medical instruments and other medical equipment/supplies, buildings (e.g., structures suspected of mold contamination), nutritional supplements, cosmetics, and personal care products.

Samples may comprise blood and/or other liquids or liquefied samples of biological origin or suspected of containing biological material, including, e.g., biological materials obtained from cells, tissues, bacteria, and/or viruses. In some examples, the sample may comprise urine, mucus, bile, lymph, sweat, saliva, gastric acid, or peritoneal fluid, among other examples of biological fluids. Further, samples may be obtained directly from a subject (e.g., clinical samples) or may be obtained indirectly from a subject or derived from a clinical sample (e.g., derived from cells in culture, cell supernatants, or cell lysates). In some aspects, a sample may be processed after being obtained from a subject and prior to analysis. For example, a sample may be processed by removing cell-free material, concentrating a portion of the cells present in the sample, concentrating all cells present in the sample, and/or lysing some or all cells in the sample. In addition or alternatively, a sample may be treated with one or more reagents, solubilized, and/or enriched for certain components. Enrichment of a sample may include, for example, concentrating one or more constituents of the sample to assist in detection, analysis, and/or identification of that constituent or another constituent of the sample.

Targets

The term “target” as used herein includes, but is not limited to, chemical and biochemical species comprising at least one natural or synthetic nucleic acid (e.g., DNA and/or RNA) or fragment thereof, including an oligonucleotide. Exemplary targets include, for example, natural and synthetic oligonucleotides, including single-stranded nucleic acids and oligonucleotides. Targets suitable for detection in the systems and methods herein may comprise, for example, one or more of the following or a fragment thereof: DNA, RNA, products of a polymerase chain reaction (PCR), genomic DNA (gDNA), messenger RNA (mRNA), microRNA (miRNA), pre-mature miRNA, mature miRNA, artificial miRNA, ribosomal RNA (rRNA), non-coding DNA, non-coding RNA, nucleic acid biomarkers, and synthetic aptamers. As discussed below, a single target may be detected and analyzed, or multiple targets may be detected and analyzed simultaneously.

In some aspects of the present disclosure, the target nucleic acid may comprise from 15 to 50 nucleotides. For example, the target nucleic acid may comprise from 18 to 50 nucleotides, from 16 to 40 nucleotides, from 17 to 35 nucleotides, from 18 to 30 nucleotides, from 19 to 25 nucleotides, or from 20 to 24 nucleotides. For example, the target nucleic acid may comprise 15, 16, 17, 18, 19, 20, 21, 22, 23, 24, 25, 26, 27, 28, 29, or 30 nucleotides. In some aspects of the present disclosure, the target nucleic acid may comprise more than 50 nucleotides, such as from 51 to 60 nucleotides, from 61 to 75 nucleotides, from 76 to 90 nucleotides, or from 91 to 100 nucleotides. In some examples, the target may comprise an RNA molecule or a fragment of an RNA molecule comprising from 15 to 50 nucleotides, e.g., from 18 to 35 nucleotides, or from 20 to 22 nucleotides. In other examples, the target may comprise a DNA molecule or a fragment of an DNA molecule comprising from 15 to 50 nucleotides, e.g., from 18 to 35 nucleotides, or from 20 to 22 nucleotides.

In some examples, the target(s) may include one or more small RNA or DNA molecules or fragments of RNA or DNA molecules obtained from the extraction of a biological fluid, such as blood or other biological fluid, such as fluid from tissue (e.g., plasma and formalin-fixed and paraffin-embedded tissues). The target(s) may comprise one or more nucleic acid fragments complexed with a binding protein, an antibody, or an aptamer bound with a target protein, or a nucleic acid fragment complexed with a pharmaceutical agent or other chemical compound. In some examples, the target(s) may include a sequence with one or more mutations, single-nucleotide polymorphism, or one or more chemical modifications, such as methylation and/or phosphorylation.

The target may be associated with one or more health conditions, such as a disease. The disease or other health condition may be genetic or environmental in origin, or associated with one or more pathogens, such as bacteria, viruses, fungi, or protozoa. For example, the target may serve as a biomarker, e.g., a chemical or biochemical indicator associated with a biological process, a pathogenic process, and/or a response to therapeutic treatment. In some aspects, the target may comprise a predictive biomarker, a diagnostic biomarker, a prognostic biomarker, or a biomarker useful for genotyping an organism. In some aspects of the present disclosure, the target may be obtained from a microbe (e.g., a nucleic acid or nucleic acid fragment of a bacterium, virus, fungus, or protozoan), may comprise a nucleic acid or nucleic acid fragment generated in response to the presence of a microbe acting as a pathogen (an infection), and/or may serve as a marker for resistance to particular antibiotic therapies. Further, the target may comprise a biomarker indicative of biological contamination, such as microbial contamination. Microbes from which target nucleic acids may be obtained include, but are not limited to, bacteria such as Escherichia coli (including, e.g., E. coli O157:H7, Enteroaggregative E. coli (EAEC), Enteropathogenic E. coli (EPEC), Enterotoxigenic E. coli (ETEC), lt/st Shiga-like toxin-producing E. coli (STEC) and Shiga toxins Stx1 and Stx2, and Enteroinvasive E. coli (EIEC)), Shigella, Salmonella Typhi, Staphylococcus aureus, Candida albicans, Klebsiella, Pseudomonas aeruginosa, Acinetobacter baumannii, Proteus, Enterobacter (including, e.g., Enterobacter cloacae complex), Serratia marcescens, Bacteroides (including, e.g., Bacteroides fragilis), Legionella, Chlamydia pneumonia, Neisseria meningitides, Streptococcus pneumonia, Clostridium, Enterococcus, Listeria monocytogenes, Streptococcus agalactiae (also known as Group B streptococcus), Streptococcus pyogenes (also known as Group A streptococcus), Candida glabrata, Candida krusei, Candida parapsilosis, Candida tropicalis, Haemophilus influenzae, Enterobacteriaceae, Klebsiella oxytoca, Cryptococcus gattii (Cryptococcus neoformans var gattii), Bordetella pertussis, Chlamydophila pneumoniae, Mycoplasma pneumoniae, Campylobacter (including, e.g., Campylobacter jejuni, Campylobacter coli, and Campylobacter upsaliensis), Clostridium difficile (including Clostridium difficile toxin A and Clostridium difficile toxin B), Plesiomonas shigelloides, Yersinia enterocolitica, and Vibrio (including, e.g., Vibrio parahaemolyticus, Vibrio vulnificus, and Vibrio cholerae); viruses such as Cytomegalovirus, Enterovirus, Herpes simplex virus 1, Herpes simplex virus 2, Herpes simplex virus 3, Human parechovirus, Varicella zoster virus, Adenoviridae (e.g., Adenovirus F 40 and Adenovirus F 41), Human coronavirus 229E, Human coronavirus HKU1, Human coronavirus OC43, Human coronavirus NL63, Human metapneumovirus, Human rhinovirus, Human enterovirus, Influenza A (e.g., Influenza A/H1, Influenza A/H1-2009, Influenza A/H3, Influenza H5N1, and/or Influenza H7H9), Influenza B, Parainfluenza 1, Parainfluenza 2, Parainfluenza 3, Parainfluenza 4, Respiratory syncytial virus (RSV), Astrovirus, Norovirus GI, Norovirus GII, Rotavirus A, and Sapovirus (e.g., Sapovirus I, Sapovirus II, Sapovirus IV, and Sapovirus V); or parasites such as Cryptosporidium, Cyclospora cayetanensis, Entamoeba histolytica, and Giardia lamblia. Nucleic acids that may serve as markers of antibiotic resistance and resistant strains may include, but are not limited to, mecA (e.g., resistance to methicillin, penicillin and/or other penicillin-like antibiotics), vanA and vanB (e.g., resistance to vancomycin), methicillin-resistant Staphylococcus aureus, (MRSA) (e.g., resistance to beta-lactam antibiotics such as penicillins and cephalosporins), and Klebsiella pneumoniae carbapenemase (KPC) (e.g., resistance to carbapenem).

Multiple nucleic acids associated with a particular health condition may be detected and distinguished from one another according to some aspects of the present disclosure. In some examples, the target or targets may be part of a collection of biomarkers associated with the health condition(s). For example, different types of cancer are associated with distinct miRNA expression profiles, which may include miRNA “families” containing miRNAs that differ from one another by one, two, or several nucleotides. MiRNAs may be released from a cancerous tumor into blood stream in a stable or relatively stable form. Circulating miRNAs are reportedly enveloped inside exosomal vesicles, and transferable and functional in the recipient cells. In some aspects of the present disclosure, detection of miRNAs may assist in early diagnosis, staging, and/or monitoring of cancer cells.

Exemplary targets that may be detected, identified, and/or quantified according to some aspects of the present disclosure include, but are not limited to, miR-155, miR-39, miR-21, miR-210, miR-182, let-7a, let-7b, and let-7c.

Probe Molecules

A probe molecule complementary to each target of interest may be used to detect the targets. The probe molecule may comprise a sequence fully complementary or partially complementary to the target of interest, e.g., such that the probe molecule may hybridize with (also described as binding to, or capturing) the target. For example, the probe molecule may include at least 4, 6, 8, 10, 12, 14, 15, 16, 17, 18, 19, 20, 21, 22, 23, 24, 25 or more nucleotide or nucleobase residues complementary to the target nucleic acid. In some examples, the probe molecule may comprise from 15 to 50 nucleotides complementary to the target, e.g., from 18 to 50 nucleotides, from 16 to 40 nucleotides, from 17 to 35 nucleotides, from 18 to 30 nucleotides, from 19 to 25 nucleotides, or from 20 to 24 nucleotides complementary to the target. For example, the probe molecule may comprise 15, 16, 17, 18, 19, 20, 21, 22, 23, 24, 25, 26, 27, 28, 29, or 30 nucleotides complementary to the target. The nucleotide or nucleobase residues complementary to the target may form a continuous sequence, or may be interrupted by one or more non-complementary nucleotide or nucleobase residues. For example, the probe molecule may comprise two or more continuous sequences complementary to a target separated by one or more nucleotide or nucleobase residues that are not complementary to the target.

In some aspects, the probe molecule may comprise an oligonucleotide comprising natural DNA nucleotides (A, T, G, C), natural RNA nucleotides (a, u, g, c), modified or derivatized DNA and/or RNA nucleotides, and/or artificial nucleotides. Exemplary artificial, modified, or derivatized nucleotides that may be used in probe molecules include, but are not limited to, locked nucleic acid (LNA) (comprising modified RNA nucleotides having a bridge connecting the 2′ oxygen to the 4′ carbon), peptide nucleic acid (PNA) (having a backbone structure comprising repeating N-(2-aminoethyl)-glycine units linked by peptide bonds), glycol nucleic acids (GNA) (having a backbone structure comprising repeating glycol units linked by phosphodiester bonds), threose nucleic acids (TNA) (having a backbone structure comprising repeating threose sugars linked by phosphodiester bonds), morpholinos, and nucleosides such as inosine, xanthosine, 7-methylguanosine, dihydrouridine, and 5-methylcytidine.

Probe molecules according to the present disclosure may comprise at least one tag, which may located at, or proximate, an end of the probe molecule. For example, the probe molecule may comprise a tag at the 3′ terminal or the 5′ terminal, or a tag at each of the 3′ terminal and the 5′ terminal of the probe molecule. In some aspects, the tag may comprise a single chain molecule of any suitable length for detection of the target. For example, the tag may have sufficient length to assist in trapping the target/probe complex in the nanopore and/or unzipping the target/probe complex during translocation through the nanopore. The tag(s) of a probe molecule may help to induce voltage-driven separation (unzipping) of the probe/target complex. Exemplary tags include, but are not limited to, polymers. In some aspects, for example, the tag may comprise an oligonucleotide such as poly(dG)ₙ, poly(dC)ₙ, poly(dA)ₙ, and/or poly(dT)ₙ, wherein n is an integer greater than 6, greater than 10, greater than 20, or greater than 30, e.g., an integer chosen from 8, 10, 12, 14, 15, 16, 18, 20, 22, 24, 26, 28, 30, 32, 34, 36, 38, or 40. In some aspects of the present disclosure, the probe molecule may comprise poly(dC)ₙ wherein n is an integer ranging from 10 to 500, such as from 10 to 300, from 10 to 100, from 10 to 50, or from 10 to 15, e.g., 10, 11, 12, 13, 14, or 15. For example, the probe molecule may comprise poly(dC)₁₀, poly(dC)₁₁, poly(dC)₁₂, poly(dC)₁₃, poly(dC)₁₄, or poly(dC)₁₅.

In some examples, the tag may comprise a charged polypeptide molecule. Other exemplary polymers include polyethylene glycol (PEG) molecules of any lengths, such as, e.g., PEG-3, PEG-8, PEG-24, PEG-30, PEG-60, PEG-80, PEG-160, and PEG-240, peptides (e.g., peptides comprising fewer than 50 residues), dextran sulfate molecules of any lengths such as 8000 kDa, cyclodexrin (alpha, beta, and gamma), maltodextrin (3-17 unit chains), and biological phosphate compounds (e.g., adenosine triphosphate and inositol triphosphate). A probe molecule may comprise one tag, two or more tags of the same type, or two or more different types of tags (e.g., a combination of PEG-8 and PEG-160, or a combination of poly(dC)₁₂ and PEG-20, or a combination of PEG-8 and a small peptide).

Without intending to be limited by theory, it is believed that the physical properties and/or chemical properties of the probe molecule may affect interaction of the probe/target complex with the nanopore, including, but not limited to, trapping of the probe/target complex in the nanopore, separation (e.g., unzipping) of the probe/target complex, and/or translocation of one or both of the target and the probe molecule through the nanopore. Thus, for example, the properties or characteristics of a probe molecule or type of probe molecule may determine the current pattern observed. Examples of properties of the probe molecule that may affect current pattern include, but are not limited to, length, size, shape, charge, chemical composition, and chemical reactivity.

These separation (e.g., unzipping) and trapping events may provide signature patterns in the current time series of the system, to distinguish interactions of the probe molecule with the target from interaction with other components in the sample, thereby assisting in selectivity and/or specificity in target detection. Each target/probe complex may provide a distinct signature pattern corresponding to an event or combination of events, which may be used to identify the target. Further, the nature of the interaction between probe molecule and/or target may affect the sensitivity of detection. For example, an increase in trapping rate or translocation rate (the number of signature patterns over time) may correspond to higher sensitivity.

The probe molecule may be positively charged, neutral, or negatively charged. In some aspects, the tag may comprise a charged polymer, such as a peptide.

Nanopores

As mentioned above, nanopores suitable for the present disclosure may define a channel (extending between the cis opening and the trans opening) for the passage of targets and/or probe molecules therethough. The nanopores may be biological or synthetic. Exemplary biological nanopores include, but are not limited to, protein nanopores that may or may not be derivatized with selected functional groups or surface species. In some aspects, the system may comprise one or more nanopores chosen from Staphylococcus aureus α-hemolysin, Mycobacterium smegmatis porin A (MspA), Bacillus subtilis phage phi29 DNA polymerase, and Escherichia coli CsgG nanopores or variants thereof, such as an α-hemolysin variant with a negatively charged ring at the trans opening of the pore, e.g., a Staphylococcus aureus α-hemolysin nanopore comprising a K131D, K131E, or K131H amino acid substitution. Exemplary and non-limiting Staphylococcus aureus α-hemolysin wild type sequences are provided herein (SEQ ID NO. 1, nucleic acid coding region; SEQ ID NO. 2, protein coding region) and available elsewhere (e.g., NCBI GenBank Accession Nos. M90536 and AAA26598). A Staphylococcus aureus α-hemolysin variant comprising a K131D substitution is provided as SEQ ID NO. 3. Synthetic nanopores may allow for the design of nanopores with a particular size, structure, and/or functionality for detection of specific nucleic acids or types of nucleic acids. Such nanopores may be formed of any suitable material or combination of materials, including, but not limited to, silicon, silicon dioxide (SiO₂), silicon nitride (Si₃N₄), molybdenum disulfide (MoS₂), aluminum oxide (Al₂O₃), boron nitride (BN), and graphene.

In some examples, the nanopore may define an ion channel having a conical or asymmetrical shape, e.g., with one opening wider than the other (e.g., a cis opening wider than a trans opening). In other examples, the nanopore may define an ion channel having a uniform cross-sectional shape, e.g., a uniform diameter. The shape of the channel may be tailored to a specific application and/or to assist in achieving a unique signature pattern for a target. For example, the shape of the channel may be designed to provide interactions between the walls of the nanopore channel and a target nucleic acid or target/probe complex, and other molecular events during translocation, providing a unique signature pattern.

The cross-sectional size of the nanopore channel may range from about 1 nm to about 6 nm, such as from about 1.1 nm to about 5 nm, from about 1.2 nm to about 4 nm, from about 1.3 nm to about 3 nm, from about 1.4 nm to about 2 nm, from about 1.2 nm to about 1.8 nm, from about 1.5 nm to about 3 nm, or from about 1.5 nm to about 2.2 nm. In some examples, the cross-sectional size of the nanopore channel may permit passage of single-stranded nucleic acids but prevent passage of double-stranded nucleic acids. In some aspects, the nanopore channel may have a minimum cross-sectional size of about 1.2 nm, about 1.3 nm, about 1.4 nm, about 1.5 nm, about 1.6 nm, about 1.7 nm, or about 1.8 nm. For example, an α-hemolysin nanopore has a cis opening about 2.6 nm in diameter, a maximum cavity diameter of about 4.6 nm, a minimum constriction diameter of about 1.4 nm, a n-barrel diameter of about 2.0 nm, and a trans opening about 2.0 nm in diameter. Further, for example, a MspA nanopore has a minimum constriction diameter of about 1.2 nm at the bottom (trans opening) of the nanopore.

Without intending to be bound by theory, it is believed that a single stranded molecule may transverse the constriction zone of a MspA nanopore, but a double-stranded species (such as, e.g., a target/probe complex) may stall. Thurs, for example, a MspA nanopore may allow for separation of the target nucleic acid and probe molecule (e.g., “unzipping” of the target/probe complex) to occur when a single-stranded tag of the probe molecule enters the constriction zone of the MspA nanopore channel ahead of the double-stranded portion of the target/probe complex. The type of event may provide a distinct electrical current signature pattern allowing for detection and identification of the target nucleic acid, and distinguishing the target from any non-target single-stranded or blunt-end double-stranded nucleic acids or other species in the sample.

In some examples, nanopore systems comprising MspA nanopores may provide signature patterns having current blockages (levels) of longer duration as compared to a similar system comprising an α-hemolysin nanopore. The interior (channel) of the MspA nanopore is naturally negatively-charged. Thus, in some examples, a mutant or variant of the MspA nanopore that has a positively-charged interior (channel) may be used. In other examples, the MspA nanopore may be used in combination with a positively-charged probe molecule, e.g., a probe molecule having a tag that includes a positively-charged peptide.

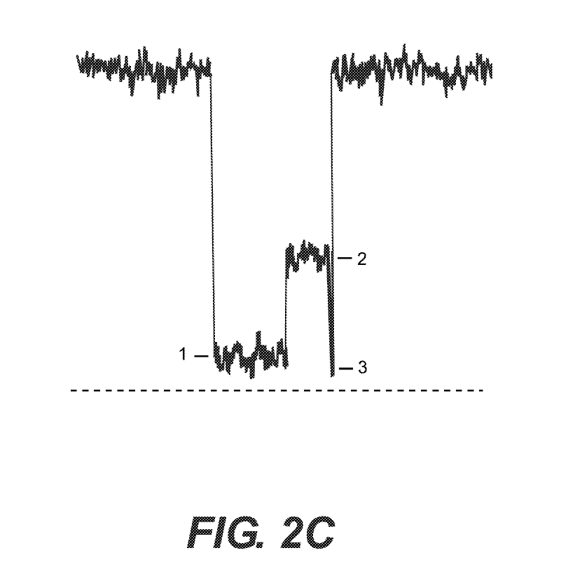

FIGS. 3A-3F illustrate some exemplary methods of preparing and incorporating nanopores into a membrane or partition. FIG. 3A shows a nanopore 300 embedded in a lipid membrane 304a, which may be prepared by creating an aperture about 150 μm in diameter in a Teflon substrate 302a by placing the wires of a spark generator on both sides of the Teflon and creating a spark through the Teflon from wire-to-wire through a spark generator set at a frequency of 15 Hz, applying the lipid membrane 304a, and then placing the nanopore 300 into the aperture. The nanopore 300 may be, for example, α-hemolysin.

Exemplary lipid materials suitable for the systems herein include, but are not limited to, 1,2-diphytanoyl-sn-glycero-phosphocholine lipid, as well as lipids made from synthetic materials. In some examples, the lipid bilayer may be prepared by folding together monolayers on opposite sides of the aperture. In some aspects, the aperture may be pretreated with hexadecane or another suitable solvent before the lipid material is applied. In another example, the lipid bilayer may be prepared by painting or otherwise applying lipids in a solvent such as n-decane directly on the aperture. In yet another example, the lipid bilayer may be prepared by liposome fusion, in which a liposome larger than the aperture may be reconstituted with the nanopore (e.g., α-hemolysin) and fused over the aperture. In yet another example, the lipid bilayer may be prepared by bringing two aqueous buffer bubbles comprising lipids and analytes together in a hydrocarbon solvent. In yet another example, the lipid bilayer may be prepared by flowing aqueous buffer over an aqueous droplet in oil.

In some aspects, apertures less than about 25 μm may be created, e.g., in a Teflon substrate or other suitable substrate material. In some aspects, the apertures may be formed by transmission electron microscopy (TEM), which may allow the size of the aperture to be controlled. Other techniques capable of forming apertures of a similar size may also be used herein. FIG. 3B illustrates an example, wherein a smaller aperture (e.g., about 500 nm in diameter) may be created in a substrate 302b of silicon nitride (Si₃N₄) via TEM. Other suitable materials for the substrate 302b include, but are not limited to, Al₂O₃ and graphene. After creating the aperture, a lipid membrane 304b may be applied by any of the methods discussed herein, and a nanopore 300 such as α-hemolysin applied to the lipid membrane 304b. The smaller size of the aperture may allow for a smaller lipid bilayer 304b as compared to the example of FIG. 3A, which may increase the stability of the nanopore 300.

In some examples, lipid membranes may be polymerized after a nanopore such as α-hemolysin has been inserted, e.g., to induce crosslinking. UV-sensitive compounds suitable for polymerization may include, but are not limited to, styrene and divinylbenzene. The UV-sensitive compounds may be mixed with lipids before being applied to a substrate. After forming the lipid membrane and inserting the nanopore, UV light may be applied, e.g., with a UV flashlight or other UV light source.

FIG. 3C illustrates an example wherein an aperture about 150 μm in diameter may be created in a Teflon substrate 302c with a spark, and a lipid membrane 304c applied to the aperture. A nanopore 300 such as α-hemolysin may be placed into the aperture, and the nanopore 300 stabilized in the lipid membrane 304c by exposure to UV radiation, e.g., to chemically crosslink the lipids with the nanopore 300. FIG. 3D shows another example, wherein TEM may be used or a smaller aperture, e.g., about 500 nm in diameter, created in a Si₃N₄ substrate 302d. A lipid membrane 304d may be applied to the aperture, and a nanopore 300 such as α-hemolysin added to the aperture. UV radiation may be applied to crosslink lipids in the membrane 304d with the nanopore 300 to stabilize the nanopore 300 in the membrane 304d.

Some nanopore systems according to the present disclosure may not comprise a lipid bilayer. In some aspects, for example, the nanopore system may comprise a solid state material. FIG. 3E shows an example wherein an aperture 310 may be formed in a substrate 302e of solid-state material, wherein the aperture 310 may be similar in shape and size to the interior of a biological nanopore such as α-hemolysin. For example, the aperture 310 may have a minimum cross-sectional size of less than 2 nm, e.g., about 1.2 nm, about 1.3 nm, about 1.4 nm, or about 1.5 nm. Such nanopore systems may provide alternatives to lipid bilayers and biological nanopores such as α-hemolysin. An aperture of about 1.5 nm in a solid-state material may be sufficient to distinguish a probe molecule from a double-stranded target/probe complex. Exemplary solid-state materials for such nanopores may include, but are not limited to, Si₃N₄, graphene, and Al₂O₃. In some aspects, TEM followed by chemical modifications of the surface may be used to create the solid-state pores.

In some aspects, the biological and the solid-state approaches may be combined. For example, FIG. 3F illustrates an example prepared by incorporating a biological nanopore 300 such as α-hemolysin directly into a solid substrate 302f with a suitably-sized aperture, e.g., having a cross-sectional size ranging from about 5 nm to about 7 nm, which may be formed with TEM or other techniques capable of forming apertures of a similar size. The substrate 302f may comprise, e.g., Si₃N₄, Al₂O₃, graphene, among other possible solid-state materials. In some examples, scanning TEM in combination with surface modification may be used to create apertures ranging from about 5 nm to about 7 nm in graphene, which then may be coated in silicon.

Since some biological nanopores, e.g., proteins, may be unstable when inserted directly into non-lipid environments, this method may include incorporating one or more chemical modifications around the aperture to produce a physiologically-suitable environment for the biological nanopore. Various surface modifications may be used, including, but not limited to, chemically binding functionalized lipids and/or surfactants to the substrate. Further examples of chemical modifications may include adding functionalized linkers such as thiols and/or click chemistry components to covalently bind the biological nanopore into the aperture.

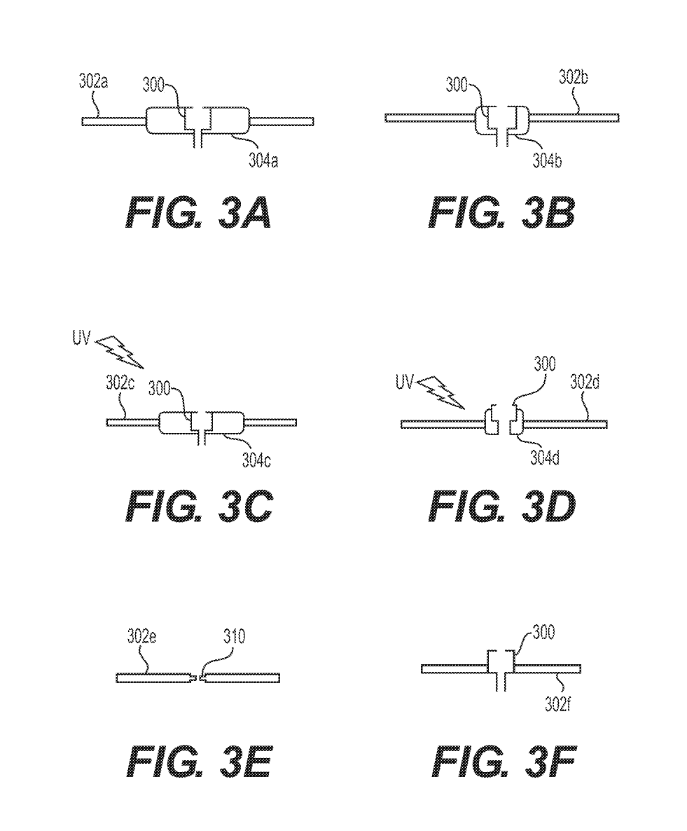

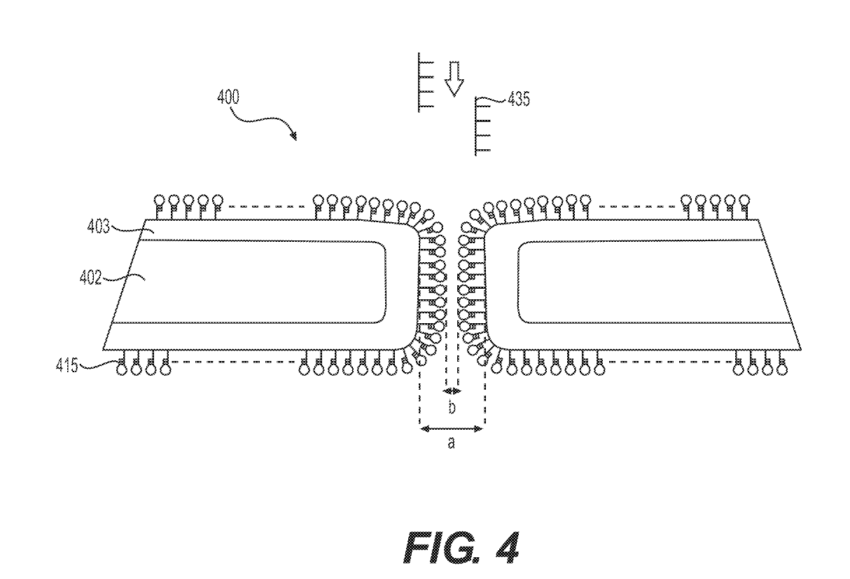

FIGS. 4 and 5A-5D illustrate further examples of solid-state nanopores, termed solid-state nanopore channels (ssNPCs), according to some aspects of the present disclosure. The ssNPCs of the present disclosure may comprise silicon-on-insulator (SOI) based nanopores. The ssNPCs may provide pore sizes similar to a biological nanopore such as α-hemolysin. In some aspects, the ssNPC nanopores may provide substantially the same limit of detection as α-hemolysin nanopores (e.g., ˜10 fM).

The ssNPC may comprise one or more DNA hairpin loops (HPLs) to create nanopore channels of a controlled size. In some examples, the ssNPCs may provide channels having a cross-sectional size of about 1.5 nm, such that the ssNPCs may be capable of acting as selective sensors for specific nucleic acid targets. In addition to creating a pore of the appropriate size, the HPL component of these ssNPCs may function similar to an oligonucleotide-based probe molecule that binds to a target as discussed above. For example, under an applied electrical field the ssNPCs may selectively transport target nucleic acids complementary to the HPL sequence through the channel, thus creating a block in current. Measuring the frequency of these current blocks may allow for quantification of the target.

In some aspects, the ssNPCs may provide a more stable and/or reusable alternative to a biological nanopore system. Further, in some examples, the single-nucleotide specificity of the ssNPCs may be similar to that of an α-hemolysin nanopore system. Some ssNPC nanopore systems according to the present disclosure may be capable of distinguishing among targets that differ by one, two, or three nucleotides. Additionally or alternatively, the ssNPC nanopore systems may be used to quantify target nucleic acids directly in cell lysates.

In some aspects, an exemplary ssNPC may be prepared as follows, with reference to FIG. 4. First, an aperture (with cross-sectional size “a”) may be created in relatively thin silicon-on-insulator (SOI) membranes 402. For example, a combination of electron beam lithography (EBL), reactive ion etching (RIE), and TEM may be used to create pore apertures of about 80 nm, which then may be decreased to a diameter of about 17 nm by controlled electron irradiation, e.g., within a limit of ±10% variation. TEM spectrographs may be used as a readout to monitor and measure the aperture size. The membranes 402 may comprise a solid-state material such as silicon, and may comprise a surface layer 403 of a different material, such as SiO₂. In some aspects, the cross-sectional size a of the aperture may range from about 16 nm to about 18 nm.

The aperture then may be functionalized to provide for a smaller channel, e.g., a nanopore channel (with cross-sectional size “b” in FIG. 4). For example, the surface of the aperture in the SOI membrane 402 may be modified and coated (partially or fully coated) with selective DNA HPLs 415 to achieve smaller nanopore channels of a given diameter, such as ranging from about 1 nm to about 2 nm, e.g., nanopore channels of about 1.5 nm in diameter. In some aspects, the cross-sectional size b of the channel may range from about 1 nm to about 2 nm, such as about 1.5 nm. In some examples, nanopore channels larger than 2 nm may be prepared. The DNA HPLs may at least partially cover the surface of the aperture, as well as other surfaces of the membrane 402, as shown in FIG. 4.

In some aspects of the present disclosure, the DNA HPLs 415 may be designed or chosen based at least in part on the sequence of the target 435 of interest to be detected. In at least one example, the ssNPC may comprise 20 base pair (bp) DNA with 10 bp HPL regions, wherein the HPL regions are targeted to 10 bp single-strand target nucleic acid sequences. The DNA HPLs 415 may be incorporated onto the surface 403 of pretreated SOI-ssNPC chips to functionalize the surface 403, e.g., create nanopore channels ranging from about 1 nm to about 2 nm. If higher specificity or reduction of the channel size is desired, the number of bases in the HPLs may be altered.

The HPL sequences may be chosen to minimize the free energy change (ΔG) and/or maximize the melting temperature (Tm), e.g., in order to promote or ensure stability. In some examples, amine-modified HPL-DNA may be attached using a bilayer strategy, e.g., a bilayer comprising 3-amino-propyl-trimethoxy-silane and 1,4-phenylene di-isothiocyanate. This may create a nanopore channel less than 2 nm that functions substantially similarly to α-hemolysin in the exemplary nanopore systems discussed above, e.g., to identify targets with single base pair sensitivity. In some examples, ssNPCs of about 1.4 nm±0.5 nm in diameter may be formed.

Single strand nucleic acids typically may pass through a channel of about 1.2 nm or larger. The signals corresponding to single-strand nucleic acids may be well defined with a relatively high signal-to-noise ratio when the nanopore is close to 1.2 nm in diameter (e.g., ˜50 S/N for an α-hemolysin nanopore). If the variability in fabrication of the ssNPCs is larger than desired (e.g., larger than about 1.4 nm±0.5 nm), the size of the SOI apertures may be reduced to reduce variability. For example, the SOI apertures may be less than about 17 nm, which after functionalization, may form ssNPCs of 1.2 nm±0.5 nm in diameter. The ssNPCs greater than or equal to about 1.2 nm may act as sensor devices for target nucleic acids, whereas nanopores with smaller channels may be inactive for nucleic acid detection. Thus, the variability of “active” pores may be reduced. In some examples, the nanopore channel size and/or the variability in channel size also may be tuned by changing the length of the DNA HPLs. The pore size may be tested with conductance measurements.

In some aspects of the present disclosure, target nucleic acids to be analyzed from a sample (e.g., nucleic acid fragments from microbial species) may be maintained at a relatively low concentration and/or a relatively large number of nanopores may be employed for each sequence being detected. This may help to address potential decreased performance of the nanopore sensor, e.g., due to the DNA HPLs opening and potentially losing structure and/or activity, such as after hybridization with a target or another species of the sample. The possibility of targets hybridizing to surface HPLs (e.g., due to diffusion) may be low, such that the DNA-HPL coating may not interfere with the formation or function of the nanopore. Reactivation of the DNA-HPL may be achieved, e.g., by appropriate temperature cycling and/or by changing the background ionic concentration of the nanopore system and flushing the system.

The ssNPC nanopore systems herein may have substantially the same configuration as the α-hemolysin nanopores discussed above, wherein the SOI membrane comprising an ssNPC may be inserted in place of the lipid bilayer in the aperture of a Teflon film in which the nanopore sits in some examples discussed above (e.g., FIGS. 3A-3D). The performance parameters of the ssNPC nanopore systems herein may be determined including, e.g., sensitivity, signal-to-noise ratio, quantification in the presence of background material (e.g., other materials present in a raw sample), and the amount of blocking or background events that may affect data acquisition.

In some examples, the sensitivity or limit of detection (signal distinguishable from a blank background) of the ssNPC nanopore systems may be about 10 fM. In some aspects, the sensitivity may be increased, e.g., by using a salt gradient or incorporating positive charges on the surface of the substrate forming the nanopore channel. If the channel becomes blocked, the material within the nanopore causing the blockage may be removed by reversing voltage at regular intervals, flushing with buffers, and/or by changing the temperature. In some examples, the signal to noise ratio sufficient for detection may be about 20 or higher.

In sample purity experiments of some ssNPC nanopore systems herein, target nucleic acids may be spiked into bacterial cell lysates and quantified. The ability to distinguish the target from background may be confirmed by detecting and distinguishing background translocation events from signals associated with target nucleic acids. For example, the ssNPC nanopore system may have undesired background signals of less than −10%. In some aspects, the ssNPCs may be created such that pore selectivity does not change over time and the number of active pores remains constant or within an acceptable range of variability.

Several additional exemplary ssNPCs are illustrated in FIGS. 5A-5D. In these examples, apertures of a given cross-sectional size (labeled “a”) may be bored into a substrate comprising a solid-state material, such as Si, SiO₂, Si₃N₄, MoS₂, Al₂O₃, BN, graphene, or a combination thereof, to form channels. The interior surface of the channels may be chemically modified to create smaller nanopore channels (cross-sectional size labeled “b”) for detection of targets. In some examples, the larger cross-sectional size a may range from about 16 nm to about 18 nm, and the smaller cross-sectional size b forming the nanopore channel may range from about 1 nm to about 2 nm. Only the aperture of the channel may be chemically modified (as shown in FIGS. 5A-5D), or other surfaces of the substrate also may be chemically modified. The ssNPC nanopore systems herein may include one type or configuration of solid-state nanopore, or may comprise a combination of two or more different types or configurations of solid-state nanopores.

FIG. 5A shows an exemplary ssNPC nanopore system 500a comprising a DNA HPLs 513 that are bound to, and partially fill, a modified aperture bored in a substrate 502a, such that a nanopore channel of about 1.5 nm is formed. Under an applied electrical field, the DNA HPLs 513 may selectively bind to and transport targets complementary to the HPL sequence through the nanopore channel creating a block in current. Measuring the frequency of these current blocks may allow for quantification of the target.

FIG. 5B shows another exemplary ssNPC nanopore system 500b similar to that shown in FIG. 5A. The DNA HPLs 515 bound to the aperture of the substrate 502b may comprise poly(dC)ₙ sequences forming a nanopore channel of about 1.5 nm. In this example, the poly(dC)ₙ sequences may prevent target sequences from binding to the channel. For this type of ssNPC nanopore system, targets may be bound to a probe molecule as discussed above (e.g., a positively charged probe molecule, or other suitable probe molecule), such that the double-stranded target/probe complex may enter the opening of the ssNPC and the probe molecule separated from the target induced by the size constraints of the channel.

FIGS. 5C and 5D illustrate examples of ssNPCs without DNA-HPL surface functionalization. FIG. 5C shows a ssNPC nanopore system 500c, wherein a double-stranded DNA molecule (dsDNA) 517 may be attached to the aperture of the modified substrate 502c in place of DNA HPLs to form nanopore channels, e.g., ranging from about 1 nm to about 2 nm in cross-sectional size. In the exemplary ssNPC nanopore system 500d shown in FIG. 5D, polypeptides 519 may be attached to the substrate 502d to form nanopore channels ranging from about 1 nm to about 2 nm. For example, a negatively charged polypeptide of about 15-17 nm in length may be synthesized, e.g., with glutamate and aspartate, and used in place of DNA HPLs inside the aperture. The polypeptide may be made rigid, e.g., by incorporating suitable molecules such as polyproline and/or other small molecules or linkers, and/or by attaching the polypeptide to DNA. For the configurations shown in FIGS. 5C and 5D, a probe molecule (such as, e.g., a positively charged probe) may be bound to the target, and the target/probe complex may enter the opening of the channel for detection as discussed above.

Nanopore systems may be prepared according to the methods discussed above for a cartridge, e.g., a consumable cartridge for use in a portable detection device (discussed further below). For example, the nanopore systems may be assembled and deposited in wells of the cartridge by one or more of the following methods: 1) Insert lipid or synthetic membranes and the nanopore in solution during manufacturing, seal each well in a cartridge, ship, and then use; 2) Insert lipid or synthetic membranes and pore in solution during manufacturing, dry chamber, seal chamber, ship, and then rehydrate directly before use; or 3) Ship cartridge with pre-formed apertures (e.g., in Si₃N₄ or other suitable materials) without nanopores or membrane; insert the nanopore/membrane, e.g., by an automated instrument into which the cartridge may be inserted before the sample to be analyzed is applied.

Multiplexed Detection

The present disclosure also includes assays for detection of multiple nucleic acid targets. Multiplexed detection capacity may be achieved with instrumentation designed to run multiple assays in parallel and/or quantifying multiple nucleic acids in one assay. To build a multiplexed chip or cartridge (e.g., comprising multiple nanopore systems run parallel), nanopore fabrication may follow the same procedures as discussed above, but used to generate multiple nanopore systems.

For some systems herein, multiple nanopores may allow for more rapid data collection. When utilizing synthetic nanopores like the ssNPCs described above, variability in pore size may be addressed by reducing the variability in size to 1.0 nm±0.5 nm. Thus, the nanopores defining a channel with a minimum cross-sectional size of about 1.2 nm or larger may fall within a specified size range, while nanopores smaller than 1.2 nm may be inactive for nucleic acid detection and not included in data analysis.

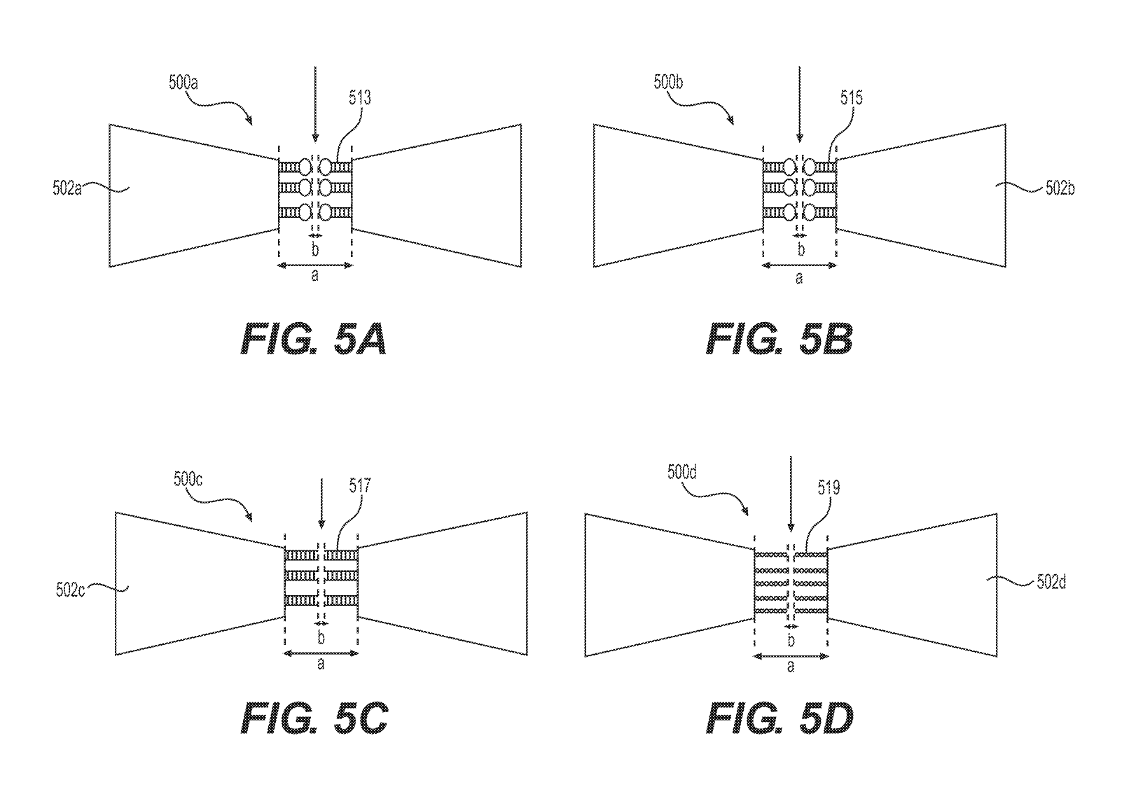

In some aspects of the present disclosure, multiple targets may be quantified in one nanopore. FIG. 6 shows a schematic comparing the current time-series obtained for probe molecules 611, 612, 613, 614 passing through the channel of the same nanopore 600, the probe molecules 611, 612, 613, 614 having different types of tags attached to the backbone of the probe molecules, e.g., respective tags 1, 2, 3, and 4. The tags 1, 2, 3, 4 may be attached along any portion of the backbone of the probe molecules 611, 612, 613, 614, e.g., attached to an internal residue in the single-stranded region of the probe molecules. As shown, the probe molecules 611, 612, 613, 614 may hybridize to a target 620 to form a target/probe complex, which then may enter an opening of the nanopore 600 embedded in a partition 602. Each tag 1-4 may result in a different signature current pattern, e.g., depending on such characteristics as length, size, shape, charge, chemical composition, and chemical reactivity as mentioned above. In some examples, the shape or configuration of the tag may cause the probe molecule to occupy more or less space in the nanopore, which in turn, may result in a larger and/or longer blockage of current.

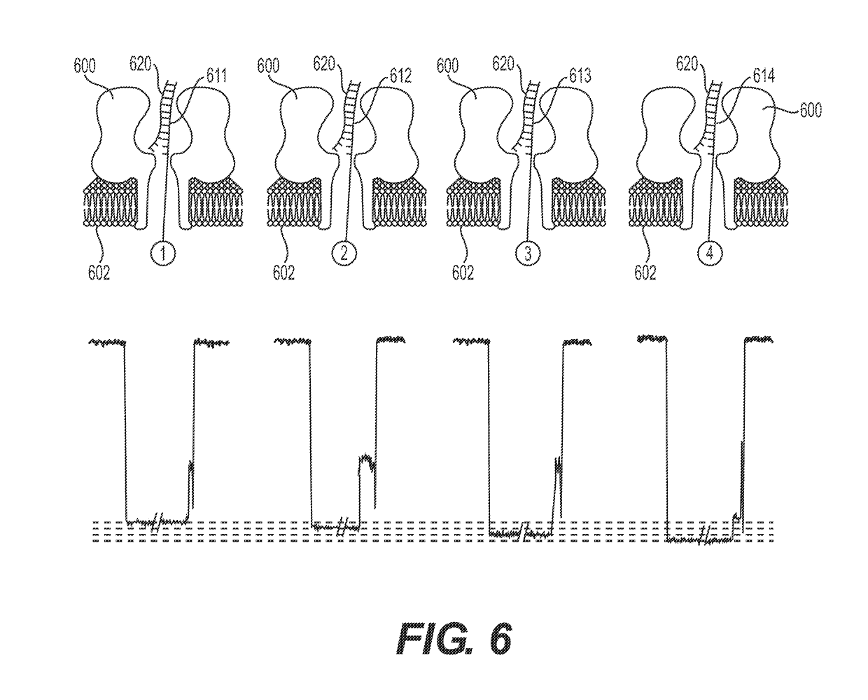

Further, in some examples herein, the probe molecule may include multiple tags that provide a unique signature pattern. In some aspects, multiple tags may serve as a barcode of the probe molecule. FIGS. 7A and 7B show examples of probe molecules 715, 717 hybridized to a target nucleic acid 708, 709, respectively, as well as a current time-series and signature pattern corresponding to interaction of the respective target/probe complexes with a nanopore. The targets 708, 709 may differ by only 1-5 nucleotides. For example, the targets 708, 709 may have sequences that are identical other than a difference in only one nucleotide,

As shown, probe molecule 715 includes two different tags, labeled a and b, attached at spaced intervals along the backbone of the probe molecule 715. Similarly, probe molecule 717 includes two different tags b, c spaced apart by at least 100 bp on a poly(dN)ₙ tail of at least 200 bp. In some aspects, the tags a-c may comprise different types and/or lengths of polymers, such as PEG molecules of different lengths. In at least one example, tag a may comprise PEG-80, tag b may comprise PEG-240, and tag c may comprise PEG-160. In some aspects, the tags a-c may comprise nucleic acids with sequences complementary to the probe molecules 715, 717, such that the probe molecules 715, 717 comprise relatively short double-stranded regions where each tag is attached.

While the signature patterns of FIGS. 7A and 7B both include 5 levels, they are distinguishable from one another due to variations in the magnitude and/or duration of the levels, e.g., resulting from the differences in the types of tags a-c and location of each tag on the respective probe molecules 715, 717. Such unique signature patterns may provide for a multiplexed approach for distinguishing among targets having similar sequences.

Quantification

The frequency of signature patterns observed for a target nucleic acid may be used to determine the concentration of that target in a sample. Quantification of a target nucleic acid in a sample with a nanopore system as disclosed herein may be performed by spiking aliquots of the sample with different, known concentrations of the target nucleic acid along a linear range as controls. The frequency of signature patterns (number per unit time) for each control then may be measured, as well as the frequency of signature patterns in the unspiked sample (for the target nucleic acid of unknown concentration), e.g., utilizing a multiplexed detection system. A plot may be prepared of nucleic acid concentration vs. frequency of signature patterns, and a linear regression performed to obtain a best-fit line. The best-fit line then may be used to determine the concentration of the target in the sample given its frequency of signature pattern.

Additionally or alternatively, the concentration of a target may be determined by characterizing the performance of each target/probe complex of interest in a given nanopore system within a range of different concentrations of the target. That information may be used to calculate a rate constant (Kₒₙ) relating the concentration of the target nucleic acid ([NA]) with the frequency of signature patterns (f_{sig}) for that target: f_{sig}=Kₒₙ*[NA]. A predetermined Kₒₙ then may be used to calculate [NA] for an unknown sample in an experimental situation given the measured f_{sig} ([NA]=f_{sig}/Kₒₙ).

Exemplary Assays

Nanopores may allow for the detection and quantification of miRNAs in a portable format. But miRNAs represent only a portion of nucleic acid biomarkers useful in medical diagnosis. Larger nucleic acids that have value as diagnostic biomarkers include, but are not limited to, genomic DNA, messenger RNAs, and for microbial diagnostics, ribosomal RNAs (rRNAs). Many detection methods, including prior nanopore-based methods, are not well equipped to test for these larger nucleic acids in a rapid and highly-sensitive format.

The systems and methods herein may provide a portable and/or stable platform to allow for the detection of any nucleic acid of interest. In some aspects, for example, a target nucleic acid may be cut into one or more smaller target nucleic acid fragments for analysis with the nanopore systems herein. Thus, the larger target nucleic acid may be a parent to the target nucleic acid fragments. This may be appropriate, for example, for nucleic acids comprising more than 30 bp (more than 30 nucleotides in length), more than 40 bp (more than 40 nucleotides in length), or more than 50 bp (more than 50 nucleotides in length), when the secondary structure of the nucleic acid may impair translocation of the nucleic acid through the nanopore. In some examples, the parent nucleic acid may comprise 100 or more nucleotides in length, e.g., from 100 to 2000 nucleotides (or from 100 to 2000 bp), such as from 200 to 1800 nucleotides, from 300 to 1700 nucleotides, or from 400 to 1700 nucleotides, or from 500 to 1600 nucleotides in length.

The target nucleic acid fragments may comprise, for example, from 15 to 25 nucleotides in length, or from 16 to 22 nucleotides, e.g., 15, 16, 17, 18, 19, 20, 21, 22, 23, 24, or 25 nucleotides in length. Target nucleic acids within this size range may be suitable for establishing a binding affinity with a probe molecule that allows for nanopore-assisted separation of the target/probe complex within a reasonable timeframe for analyzing the effect on the current measured for the nanopore system. It should be noted that the nanopore systems herein may be used for analysis of nucleic acid targets comprising more than 25 nucleotides in length, however.

In some aspects, an enzyme such as endonucleases or exonucleases, e.g., a ribonuclease (RNase) or restriction enzyme, may be used to obtain the smaller target nucleic acids. Exemplary RNases that may be used herein include, but are not limited to, RNase A, RNase 1, RNase 1f, RNase III, RNase L, RNase P, RNase PhyM, RNase T1, RNase T2, RNase U2, RNase V, and RNase H. In some aspects of the present disclosure, a mixture of RNases may be used, or different types of RNases may be used in different steps of a given assay. RNase A, RNase 1, RNase 1f, RNase P, RNase PhyM, RNase T1, RNase T2, and RNase U2 digest single-stranded RNA molecules. For example, RNase A cleaves the 3′-end of unpaired cytosine (C) and uracil (U) residues, RNase 1 cleaves internal phosphodiester RNA bonds on the 3′-side of pyrimidine bases, RNase 1f cleaves RNA dinucleotide bonds, and RNase T1 cleaves RNA after guanine residues. RNase H digests double-stranded DNA/RNA molecules by cleaving the 3′-O—P bond of the hybridized RNA. RNase III cleaves double-stranded RNA. RNase V hydrolyzes poly(A) and poly(U) sequences, forming oligoribonucleotides and ultimately 3′-AMP. Other techniques for obtaining smaller target nucleic acids may include, but are not limited to, type I, II, III, and IV restriction enzymes; meganucleases, zinc finger nucleases (ZFNs), transcription activator-like effector nucleases (TALENs), and the CRISPR-Cas system.

In some aspects, protein complexes may be used, wherein the protein complexes bind to and/or stabilize hybridized RNA/DNA molecules (e.g., target/probe complexes) to provide further protection from RNases during the enzymatic processes described herein, e.g., by degrading nucleic acids flanking a target nucleic acid sequence and/or background nucleic acids. Such protein complexes may bind to an RNA/DNA target/probe complex and thereby prevent degradation of the target nucleic acid from any RNase. Exemplary protein complexes include, but are not limited to, p19 and TAL effector.

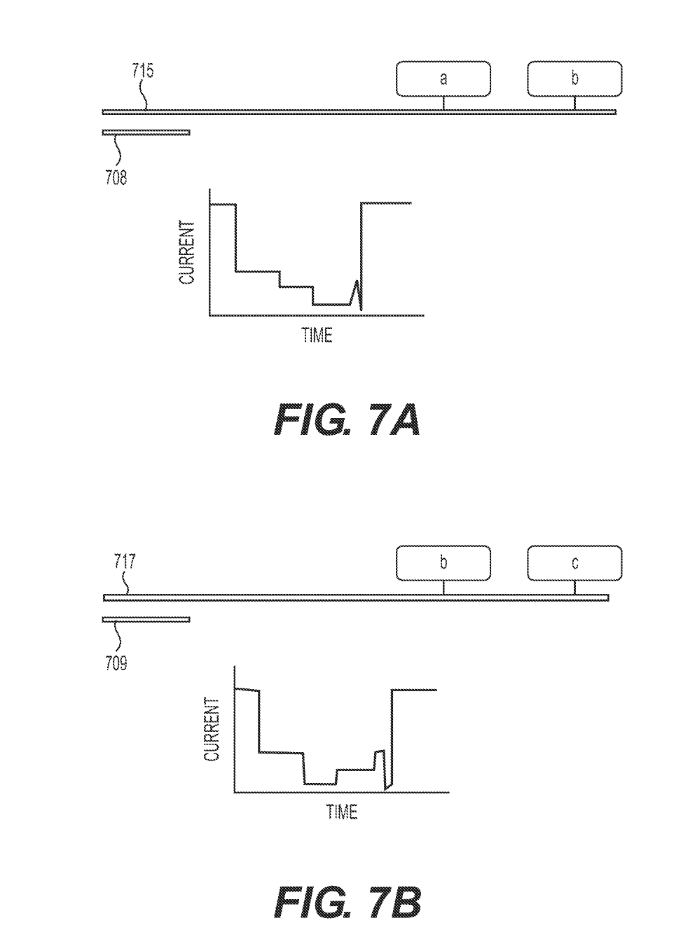

Several exemplary upstream processing assays or assay protocols that may be included in some assays herein are shown schematically in FIGS. 8-10. FIG. 8 shows a flow diagram for an exemplary assay using enzymatic digestion. The assay may include adding a probe molecule 815 to a relatively long target RNA molecule 810 (e.g., comprising more than 30 bp, e.g., more than 30 nucleotides in length). The probe molecule 815 may be partially or fully complementary to a specific portion 812 (e.g., a sequence of 15 to 25 nucleotides) of the RNA molecule 810, such that it hybridizes to that portion 812 of the RNA molecule 810.

Next, an enzyme or enzyme mixture may be added to degrade any unbound, single-stranded portions of the RNA molecule 810. The enzyme or enzyme mixture may comprise one or more of RNase A, RNase 1, RNase 1f, RNase P, RNase PhyM, RNase T1, RNase T2, or RNase U2. For example, the enzyme mixture may comprise RNase A and RNase T1. The hybridized target/probe complex 835 formed by the probe molecule 815 and target sequence 812 may remain intact. Thus, for example, the probe molecule 815 may “protect” the RNA sequence 812 of interest from enzymatic digestion of the flanking RNA sequences. The enzyme(s) also may degrade other background (non-target) RNA molecules and fragments that may be present in the sample. The target-probe complex 835 then may be detected and quantified with a nanopore system as discussed above. The digestion of non-target nucleic acids may help to reduce signal due to non-target nucleic acids passing through the nanopore (e.g., background signal, or background noise).

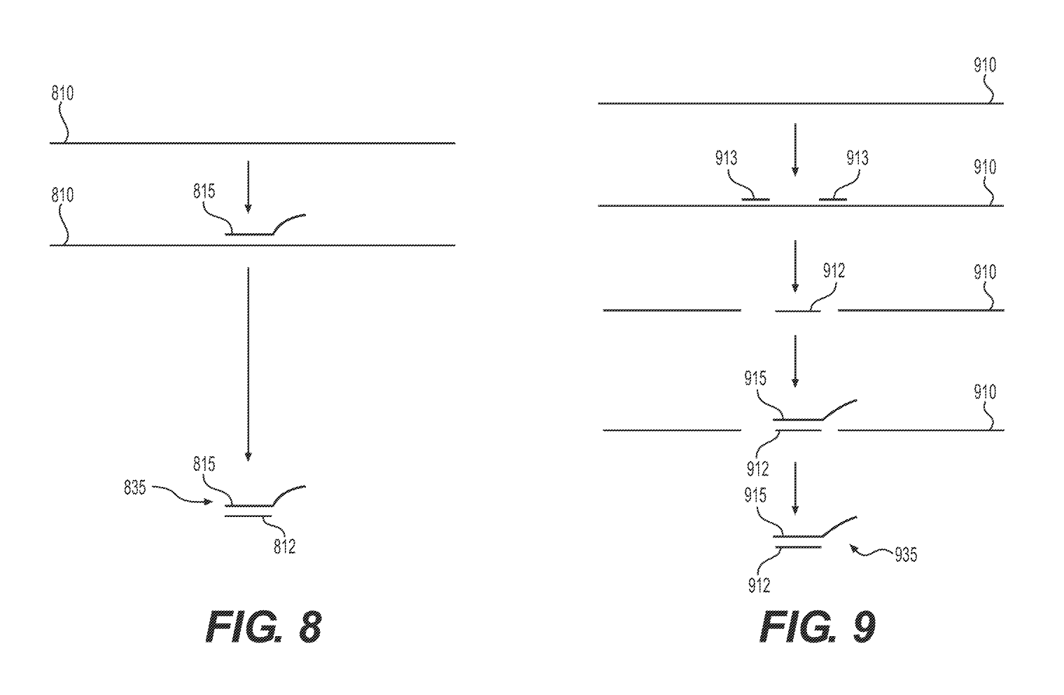

In another exemplary assay illustrated in FIG. 9, DNA probe molecules 913 may be added to a relatively long target RNA molecule 910 (e.g., comprising more than 30 bp). The DNA probe molecules 913 may be partially or complementary to portions of the RNA molecule 910 flanking a sequence of interest (the sequence of target 912), such that the DNA probe molecules 913 hybridize to the flanking regions. Thus, for example, one of the DNA probe molecules 913 may have a sequence partially or fully complementary to a sequence of the RNA molecule 910 flanking the 3′ end of the target 912, and the other DNA probe molecule 913 may have a sequence partially or fully complementary to a sequence of the RNA molecule 910 flanking the 5′ end of the target 912. After the DNA probe molecules 913 bind to the RNA molecule 910, RNAse H may be added to degrade the double-stranded regions of the RNA molecule 910 formed by hybridization of the DNA probe molecules 913. RNAse H also may digest other background (non-target) double-stranded molecules and fragments that may be present in the sample. The target RNA sequence 912 may remain intact as a separate fragment of the original RNA molecule 910.

Next, a second probe molecule 915 partially or fully complementary to the target RNA fragment 912 may be added to bind to the target RNA fragment 912 and form a target/probe complex 935, which then may be detected and quantified with a nanopore system as discussed above. In some examples, an enzyme or enzyme mixture may be added prior to analysis in the nanopore system to degrade any remaining unbound, single-stranded portions of the RNA molecule 910, which may help to reduce signal due to non-target nucleic acids (e.g., background signal, or background noise). For example, the enzyme mixture may comprise RNase A and RNase T1.

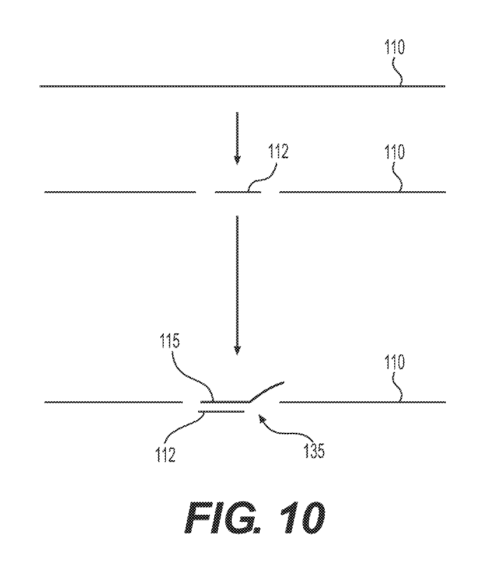

In yet another exemplary assay, shown schematically in FIG. 10, a relatively long target DNA molecule 110 (e.g., comprising more than 30 bp) may be cut into smaller target DNA fragments 112, e.g., by restriction enzymes or genome-editing nucleases such as meganucleases, zinc finger nucleases (ZFNs), transcription activator-like effector nucleases (TALENs), or the CRISPR-Cas system. The target DNA fragments 112 then may be combined with a probe molecule 115 (e.g., a PNA probe molecule or LNA probe molecule) able to outcompete the native complementary DNA strand in order to hybridize to the target DNA fragment 112. The resulting target/probe complex 135 may be detected and quantified with the nanopore systems as discussed above.

An exemplary assay in accordance with the present disclosure may comprise the following sequence of steps: (1) obtain a blood sample from a subject; (2) lyse the blood cells of the sample, and isolate pathogen cells; (3) lyse the pathogen cells, and isolate nucleic acids; add one or more probe molecules targeted to the nucleic acid sequence of interest, and then cut nucleic acids to produce target/probe complexes; then (4) detect the target/probe complexes with a nanopore system and analyze the signature patterns recorded. In some aspects, step (2) may be performed in about 5 minutes, step (3) may be performed in about 20 minutes, and step (4) may be performed in about 10 minutes.

In another exemplary assay, the following steps may be performed: (1) obtain a blood sample from a subject; (2) lyse the blood cells of the sample, and isolate pathogen cells; (3) lyse the pathogen cells, and isolate whole rRNA; add one or more probe molecules targeted to the rRNA sequence of interest, and then add RNase to cut the whole rRNA to produce target/probe complexes and digest any remaining single-stranded (unbound) rRNA; then (4) detect the target/probe complexes with a nanopore system and analyze the signature patterns observed to quantify the target rRNA of interest. In some aspects, step (2) may be performed in about 5 minutes, step (3) may be performed in about 20 minutes, step (4) may be performed in about 10 minutes, and step (4) may be performed in about 10 minutes.

In yet another exemplary assay, the following steps may be performed: (1) obtain a urine sample from a subject; (2) concentrate the cells in the sample; (3) isolate RNA in the sample; (4) add one or more probe molecules targeted to the RNA sequence of interest, and then cut the RNA to produce target/probe complexes and digest any remaining single-stranded (unbound) rRNA; (5) detect the target/probe complexes with a nanopore system; and (6) analyze the signature patterns observed to quantify the target rRNA of interest. In some aspects, step (2) may be performed in about 5 minutes, step (3) may be performed in about 30 minutes, step (4) may be performed in about 10 minutes, step (5) may be performed in about 10 minutes, and step (6) may be performed in about 15 minutes.

In at least one example, detection with a nanopore system may comprise at least two steps: (1) first adding a probe molecule to a sample, wherein the probe molecule binds to a target of interest in the sample to form a target/probe complex; and then (2) adding the sample to the cis or trans chamber of a nanopore system comprising a nanopore with an inner minimum cross-sectional size ranging from about 1.2 nm to about 1.8 nm. For example, the nanopore may be an α-hemolysin protein inserted into a lipid membrane serving as a partition between the cis chamber and the trans chamber (see, e.g., FIG. 1). A voltage may be applied across the partition to draw charged and/or neutral target/probe complexes toward the nanopore. The voltage may range from about 80 mV to about 200 mV, such as from about 90 mV to about 180 mV, or from about 100 mV to about 150 mV, e.g., about 100 mV, about 120 mV, about 140 mV, or about 150 mV.

In some examples, the sample may be added to the cis chamber of the nanopore system. The applied voltage may induce negatively-charged and/or neutral entities to pass through the nanopore channel, from the cis chamber into the trans chamber. To assist in drawing the target/probe complex toward the nanopore and/or moving the probe molecule through the nanopore channel, the probe molecule may be negatively-charged. For example, the probe molecule may include one or more negatively-charged tags. Any suitable negatively-charged polymers or other chemical species or functional groups may be used. In some examples, the probe molecule may comprise a single-stranded oligonucleotide with a poly(dC)ₙ tag attached to the 3′ end, the 5′ end, or both the 3′ end and the 5′ of the oligonucleotide, wherein n is an integer ranging from 10 to 15, e.g., poly(dC)₁₂. The poly(dC)ₙ tag(s) may increase the strength of the negative charge on the oligonucleotide probe molecule. Cis-to-trans signature patterns include, but are not limited to, the types of 2-level and 3-level signature patterns discussed above, and shown in FIG. 2.

In other examples, the sample may be added to the trans chamber of the nanopore system. The applied voltage may induce positively-charged and/or neutral entities to pass through the nanopore channel, from the trans chamber into the cis chamber. To assist in drawing the target/probe complex toward the nanopore and/or moving the probe molecule through the nanopore channel, the probe molecule may be positively-charged. While not intending to be bound by theory, it is believed that the probe/target complex may be selectively trapped using a probe molecule carrying a positive charge under an appropriate voltage, while negatively-charged non-target oligonucleotides may be prevented or inhibited from entering the nanopore. In some aspects, a positively-charged probe molecule may be used in combination with a nanopore having a negative charge, including, but not limited to, a negatively-charged residue at the trans opening of the nanopore (such as, e.g., α-hemolysin comprising a K131D mutation). Advantages to certain aspects of a trans-to-cis method of detection may include decreased background signals due to non-target nucleic acids, and/or the ability to analyze raw (unprocessed) samples, which may include non-target nucleic acids that otherwise may interfere with detection. For example, translocation of small, positively-charged polymers such as free peptides and small molecules may be distinguished from the target nucleic acid by the magnitude and/or duration of the current blockages caused by these small molecules.

The probe molecule may comprise, for example, a peptide nucleic acid (PNA), optionally with one or more positively-charged tags. Further, for example, the probe molecule may include a DNA molecule comprising one or more positively-charged tags. Any suitable positively-charged polymers or other chemical species or functional groups may be used. In some examples, the probe molecule may comprise a positively-charged polypeptide molecule, which may include two, three, four, or more amino acid residues with a positive charge. The probe molecule may include a sufficient number of positively charged residues to provide a net positive charge when hybridized to a target oligonucleotide. Trans-to-cis signature patterns include, but are not limited to, trapping events, including 1-level and 2-level signature patterns. For example, a 1-level signature pattern may correspond to a trapping event wherein the tag of a probe molecule forming a target/probe complex enters the nanopore channel, stalls, and then exits the channel to return to the trans chamber of the nanopore system. Further, for example, a 2-level signature pattern may correspond to separation of the target nucleic acid from the probe molecule (“unzipping” of the target/probe complex), wherein the tag of the probe molecule enters the nanopore channel and stalls (level 1), then separates from the target and translocates to the cis chamber while releasing the free target to the trans chamber.

Current may flow through the nanopore as ions. In some examples, the current may flow as Cl⁻ ions from a KCl solution in both the cis and trans chambers, e.g., a 1M KCl solution. Other electrolyte solutions and concentrations may be used and are contemplated herein, such as a NaCl solution ranging from about 0.5 M to about 2 M, or a KBr solution ranging from about 0.5 M to about 2 M, among other examples. In some examples, the cis and trans chambers may have different molarities, providing a concentration gradient across the partition (e.g., a cis/trans or trans/cis gradient of about 3 M/1 M KCl). The different salt concentrations on either side of the nanopore may help to increase the rate of detection by creating a positive net charge around the nanopore opening that enhances the electric capture field, resulting in increased capture rate of molecules in the nanopore.

When a molecule (e.g., a probe molecule, a target, another single-stranded nucleic acid molecule, or a small molecule) moves through the nanopore, the current flow may be interrupted causing a block in the electrical signal measured across the nanopore. In some examples, the amount of current measured across an open, unblocked nanopore (base current) may range from about 50 pA to about 200 pA, depending on the applied voltage, such as from about 80 pA to about 180 pA, or from about 100 pA to about 150 pA, e.g., a current of about 80 pA, about 90 pA, about 100 pA, about 110 pA, about 120 pA, about 130 pA, about 140 pA, about 150 pA, about 160 pA, about 170 pA, about 180 pA, about 190 pA, or about 200 pA. The blocking events (levels) of a signature pattern may have a current ranging from 0 to 99% of the base current. In some examples, a level of a signature pattern may be about 25%, about 50%, or about 75% of the base current.

As discussed above, the target/probe complex may be distinguished from a block of current due to the probe molecule alone, the target alone, or other background molecules. For example, the probe molecule and the target may pass through the channel at a faster rate (causing a shorter block of current) than the target/probe complex, e.g., as the probe molecule first un-anneals from the target before completing translocation through the channel. In some examples, the signal measured from an oligonucleotide translocation blocking event may range from about 140 pA to about 180 pA at a 150 mV potential and 1M KCl in both the cis chamber and trans chamber.

The nanopore systems disclosed herein may be used in assays for obtaining diagnostic information on a particular illness, such as a bacterial infection. For example, the nanopore systems may be used to detect and quantify microbial rRNA to identify microbe(s) associated with an infection or biological contamination, or for other clinical or non-clinical applications (see, e.g., Examples 1 and 2).

The methods disclosed herein for detection of targets to obtain diagnostic information may have a total assay time (including sample preparation, detection, and quantification) of less than about 4 hours, such as less than about 2 hours, less than about 90 minutes, less than about 1 hour, less than about 45 minutes, less than about 30 minutes, less than about 20 minutes, less than about 15 minutes, or less than about 10 minutes. Thus, for example, some assays disclosed herein may be useful in point-of-care treatment, in order to identify the type of disease, infection, or other health condition of a subject for prompt medical treatment. Further, some assays of the present disclosure may be useful for prompt screening for potential biological contamination, such as, e.g., testing of suspected municipal water supplies, food products, drugs, or medical equipment.

Devices

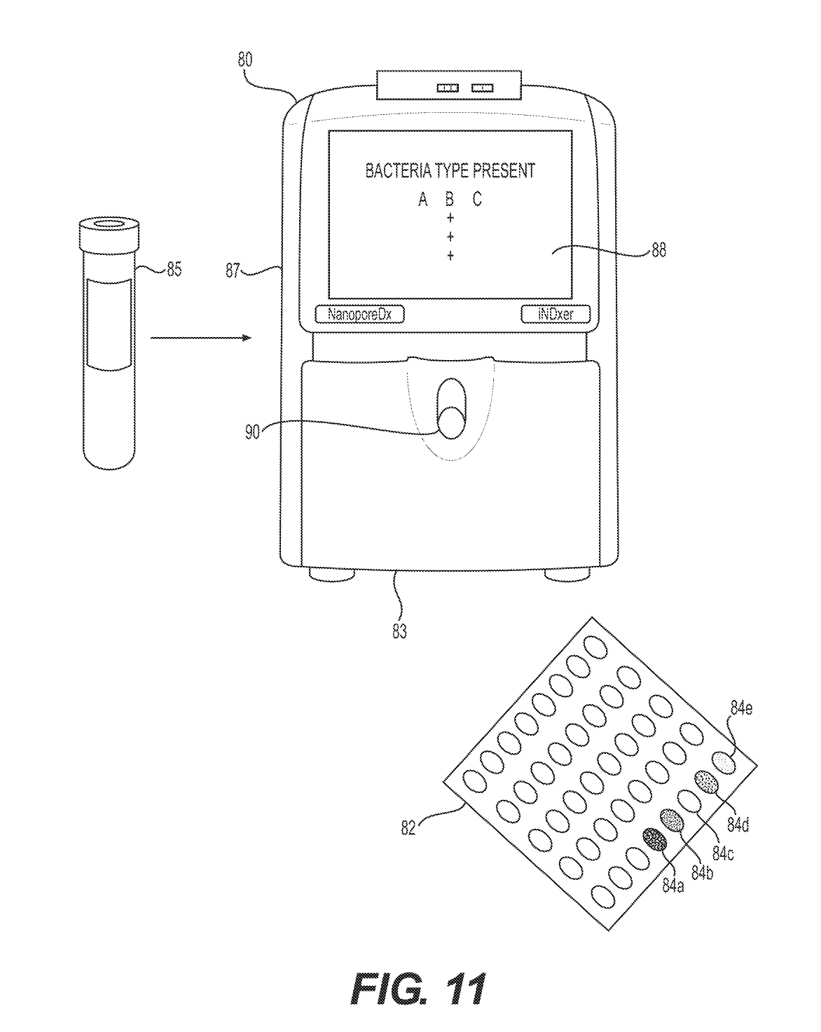

The nanopore systems disclosed herein may be incorporated into a device. For example, the device may be portable, e.g., a hand-held or small and light-weight, to allow for point-of-care functionality. An exemplary device 80 is shown in FIG. 11. The device 80 may include an inlet 87 for accepting one or more samples 85 for analysis, such as a blood sample. The sample 85 may be a raw sample (e.g., obtained directly from a subject, without processing), or may have been subjected to one or more processing steps.

In some examples, the device 80 may allow for detection of target nucleic acids in the sample 85 without a culture step or other substantial pre-processing of the sample 85. In some aspects, the device 80 may be configured to perform sample processing prior to analysis by nanopore systems in the device 80. Such in-device processing steps may comprise one or more of the following: lysing of blood or other background cells, removal of cell-free material, concentrating a portion of the cells present in the sample 85, concentrating all cells present in the sample 85, and/or lysing cells in the sample 85. Such sample processing within the device 80 may be performed with microfluidics or any other suitable techniques.

Removal of cell-free material may be performed, for example, by centrifuging cells in the sample 85, removing the supernatant, and re-suspending the concentrated cellular material in water. Additionally or alternatively, removal of cell material may be done relatively more quickly with size exclusion material, electrophoresis, isotachophoresis or other centrifuge-free manner. Cell lysis may be achieved chemically and/or physically. In some examples, cell lysis may be performed by heating at about 95° C. for about 5 minutes. In other examples, cell lysis may include bead beating, such as with magnetic beads. The beads may lyse cells through mechanical forces. In some aspects, beads may be functionalized in order to bind nucleic acids for subsequent processing, such as downstream purification and separation of nucleic acids from the beads. In some examples, chemical reagents may be used in conjunction with the processing steps discussed above, or in a separate processing step. Exemplary chemical reagents include, but are not limited to, lyzosome and detergents. In some aspects, certain detergents may be used in non-lipid based systems, e.g., only after incorporation of a polymerized membrane/partition or lipid-free membrane/partition in the system.

Another exemplary processing step may include removing particulates from the sample 85. For example, material other than nucleic acids may be removed from a cell lysate after lysis but before detection via a nanopore system. This may be done by centrifugation and keeping the supernatant, by binding nucleic acids to beads (e.g., magnetic beads) and removing unbound material, by employing nucleic acid-binding columns and changing supernatant, or by digesting unbound material with enzymes (e.g., using reagents such as lysosome and/or proteases). In further examples, the device 80 may perform the assays discussed above using RNase(s) to obtain smaller target nucleic acid fragments from a relatively longer target nucleic acid.

The sample 85 (which may or may not undergo processing as discussed above) may be analyzed with one or more nanopore systems incorporated into the device 80. In some examples, the device 80 may comprise a cartridge 82 that includes the nanopore system(s). In some examples, the cartridge 82 may be removable from the device 80, e.g., such that the cartridge 82 may be inserted into a slot 83 of the device 80 for performing an assay, and removed upon completing the assay. The cartridge 82 may be consumable, e.g., single-use, such that a new cartridge 82 may be inserted for each sample to be analyzed.

The cartridge 82 may comprise a plurality of wells 84a-84e, wherein each well may include a nanopore system as disclosed herein. Each well may include a single type of probe molecule, or a plurality of different probe molecules designed to bind to different targets (e.g., multiplex detection). In the example shown in FIG. 11, each of wells 84a, 84b, 84c, 84d, and 84e may include a nanopore system designed to detect a different target, such as targets indicative of different bacteria, different microbes, and/or different biomarkers. Each well of the cartridge 82 may have its own electrodes, such that multiple electrodes may then converge on an electron holder of the cartridge. For example, an electron holder may be located at or proximate an edge of the cartridge 82, such as the base or bottom of the cartridge 82. The electron holder may connect to a multi-channel amplifier within the device 80, which itself may be run through a processor of the device 80.

The device may include a user interface 88, such as an LED display, for showing operational parameters and/or results of the analysis, e.g., identification and/or quantification of different targets. In some aspects, the user interface 88 may include a touchscreen for accepting user input (e.g., selecting various sample processing steps, selecting which targets for analysis, etc.). The device 80 may include a power switch or on/off button for activating and deactivating power.

While FIG. 11 illustrates one exemplary device, it is understood that the present disclosure includes other types of devices, including devices comprising multiple cartridges and stationary devices.

A multiplexed cartridge may be prepared with multiple nanopores and nanopore systems having any of the exemplary configurations and characteristics disclosed herein. For multiplexed detection, for example, two or more cartridges may be inserted into corresponding slots of a device or multiplexed measuring chamber. The ssNPC nanopores, and other types of nanopores discussed above in each of the cartridges may be complementary to a distinct sequence of a target, or for biological nanopores, the probe molecule utilized in each distinct cartridge may comprise a sequence complementary to a distinct sequence of the target. Multiplexed detection also may be used to increase sensitivity, e.g., by using multiple nanopore systems targeting one target nucleic acid.

The device may record and analyze data from one cartridge or multiple cartridges to quantify a plurality of targets of a sample. In some aspects, each cartridge may comprise one or more nanopore systems for detection of one type of target. Thus, for example, eight cartridges may allow for eight different targets to be identified with the device. In some aspects, a cartridge may be configured to detect two or more different types of targets. For example, a cartridge may comprise a plurality of nanopore systems, each used for detection of a different target, or the cartridge may comprise one or more nanopore systems used to detect two or more different targets simultaneously. Each nanopore system may be given a specific location on the cartridge, e.g., a unique well position on a multi-well cartridge (see, e.g., FIG. 11).

In at least one example, the device may be configured to detect and/or quantify from 2 to 50 different targets, such as from 8 to 30 different targets, or from 16 to 20 different targets. In some aspects, a cartridge containing 50 wells may be capable of detecting 50 or more different targets. Thus, for example, a device configured to accept 10 cartridges, each including 50 wells, may be configured to screen a sample for 100 or more different targets. In some aspects, for example, about 10 nanopores may be created within an area of about 500×500 μm of a substrate, such as a cartridge. For a well having a diameter of about 3 mm, the number of nanopores many range from 1 to 60 or more, e.g., depending on the dimensions of the nanopore, the composition of the membrane, and, for synthetic nanopores, the materials used to form the nanopore. In some examples, one well of a cartridge may comprise 5-20 nanopores with a total volume ranging from about 10 nL to about 200 nL, or 20-100 nanopores with a total volume of ranging from about 200 nL to about 1 μL. In some examples, one well of a cartridge may comprise 1-5 nanopores with a total volume ranging from about 1 nL to about 10 nL, or 5-50 nanopores with a total volume ranging from about 10 nL to about 500 nL.

For patients who require treatment quickly, aspects of the present disclosure may enable physicians and other healthcare providers to diagnose illness promptly. For pathogen-related illnesses, the systems and methods herein may allow for identification of the species responsible for an infection, such that healthcare providers may administer targeted therapies to patients rather than broad-spectrum antibiotics. Embodiments of the present disclosure may help to improve patient recovery, increase patient survival, decreased use of broad-spectrum antibiotics and potential spread of antibiotic resistance, decrease costs of detection/diagnosis, decrease total treatment costs and lengths of illnesses, and/or decrease the amount of time to obtain a diagnosis.

The following examples are intended to illustrate the present disclosure without, however, being limiting in nature. It is understood that the present disclosure encompasses additional embodiments consistent with the foregoing description and following examples.

### EXAMPLES

### Example 1

Sample RNA obtained from E. coli bacteria was subjected to an enzymatic processing assay according to the present disclosure. A sample of rRNA first was isolated from whole E. coli bacteria (16s and 23s) through sucrose density gradient centrifugation. A control 22 bp rRNA representing the V3 region of the E. coli 16s was also synthesized in vitro.

For hybridization, the sample and control rRNAs were combined with a probe molecule (5′-AACTTTACTCCCTTCCTCCCCGCCCCCCCCCCCCCCC-3′, SEQ ID NO. 4) targeted to the V3 region of E. coli 16s (i.e., the control rRNA). For each of the control and the sample, 4 μL of 1 mg/mL rRNA was mixed with 2 μL of 1 μM probe molecule, 5 μL of saline-sodium citrate (SSC) buffer, and 3 μL of water. Each mixture was heated at 95° C. for 3 minutes, then left on the bench for 15 minutes.

For digestion, 2 μL of buffer (comprising 100 mM NaCl, 50 mM Tris-HCl (pH 7.9), 10 mM MgCl₂, and 1 mM dithiothreitol (DTT)) and 4 μL of a mixture of RNase A and RNase T1 (comprising 40 units of RNase A and 20 units of RNase T1) were added to each mixture resulting from the hybridization reactions. The enzyme/rRNA mixtures were incubated at 37° C. for 20 minutes, then 5 μL of 100 mM HgCl₂ was added to deactivate the enzymes.

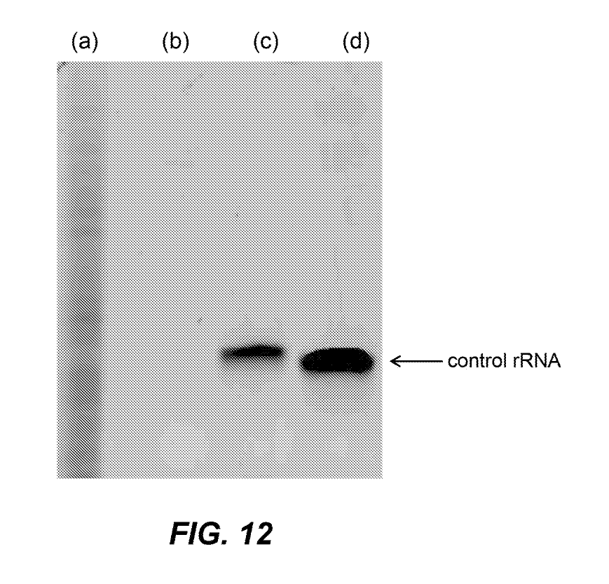

Results from gel electrophoresis are shown in FIG. 12. The gel was run for (a) the sample E. coli rRNA, (b) the sample E. coli rRNA combined with the probe molecule, without enzyme treatment, (c) the sample E. coli rRNA combined with the probe molecule, with enzyme treatment, and (d) the control rRNA combined with the probe molecule, with enzyme treatment. For the gel, a 5 μL aliquot of each of (a)-(d) was mixed with 6 μL of 2× high density loading dye (Invitrogen) and heated for 70° C. for 3 minutes, then 10 μL was run on a 15% TBE-urea gel. The gel was run at 180 v for 2 hours, and stained with GelRed™ 20 minutes before visualization. The results in FIG. 12 indicate that the RNase mixture degraded all rRNA other than the 22 bp sequence of interest bound to, and protected by, the probe molecule.

### Example 2

The specificity of probe molecules designed to target different microbial species was tested. First, 90 bp rRNA fragments corresponding to sequences from four microbial species (E. coli, Salmonella, Staphylococcus aureus, and Candida albicans) were synthesized in vitro then treated to an enzymatic processing assay according to the present disclosure.

DNA probe molecules targeting the V3 region of each species were prepared as follows:

E. coli:
(SEQ ID NO. 5)
5′-GAGCAAAGGTATTAACTTTACTC-C₃₀-3′
Salmonella:
(SEQ ID NO. 6)
5′-TGCTGCGGTTATTAACCACAACA-C₃₀-3′
Staph.:
(SEQ ID NO. 7)
5′-TACATTGTACTCATTCCAATTAA-C₃₀-3′
Candida:
(SEQ ID NO. 8)
5′-ATGTGCACAGTTACTTACACATA-C₃₀-3′

For hybridization, 4 μL of 1 μM of each 90 bp fragment was mixed with 4 μL of 1 μM of the corresponding DNA probe molecule and 8 μL of water, heated at 95° C. for 3 minutes, then left to cool on the bench for 15 minutes.

For digestion, 4 μL of buffer (comprising 100 mM NaCl, 50 mM Tris-HCl (pH 7.9), 10 mM MgCl₂, and 1 mM DTT) and 4 μL of 5 U/μl, RNase 1F were added to each mixture resulting from the hybridization reactions. The enzyme/rRNA mixtures were incubated at 37° C. for 20 minutes, then at 70° C. for 20 minutes to inactivate the enzyme.

The mixtures resulting from the enzyme reactions were added to the cis side of an α-hemolysin nanopore. To create the nanopore, 1,2-diphytanoylsn glycero-phosphocholine was dissolved in pentane and then applied to a 25 μm thin Teflon film with a 150 μm wide aperture pretreated with hexadecane. For all nanopore systems used in the examples herein, following membrane formation, an amplifier was used to assay for bilayer integrity, and only bilayer membranes with a resistance of ˜100 GΩ, a capacitance of ˜10°-200 pF, and a current noise of 1-4 pA were used.

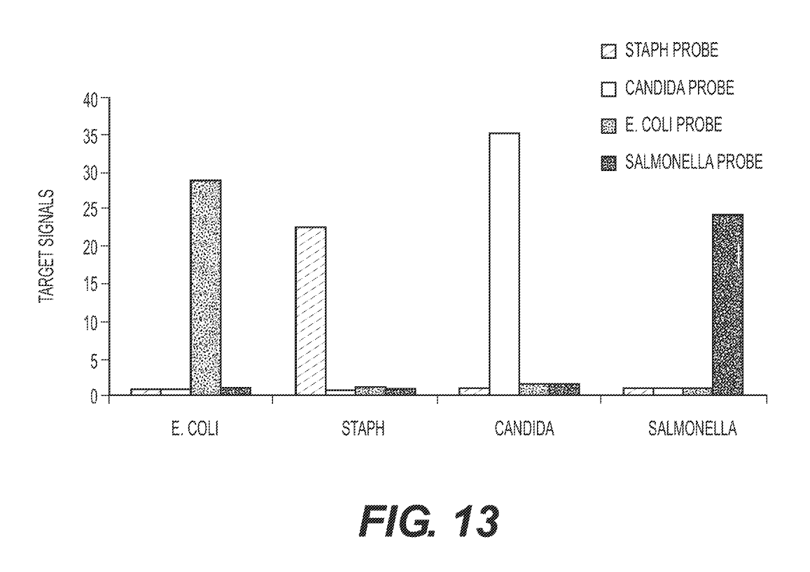

The α-hemolysin nanopore was then introduced from the cis side of the membrane. Current was recorded for about 30 minutes using an Axopatch 200B current amplifier filtered with a four-pole-low-pass Bessel filter at 5 kHz. Data was acquired through a Digidata 1440 converter at a sampling rate of 20 kHz. Ag/AgCl electrodes were used in the nanopore system. The data was then analyzed to identify 3-level signature patterns. The number of signature patterns over the 30-minute period was normalized to the single-channel time recorded (number of nanopores×the amount of time analyzed). FIG. 13 shows the number of target signals (signature patterns) observed per 30 minutes of single-channel time. Target signals were only seen above background when RNA samples were matched with the correct species-specific probe molecule.

### Example 3

A 90 bp sample of rRNA representative of E. coli bacteria containing the V3 region was synthesized in vitro and subjected to an enzymatic processing assay according to the present disclosure, as described below. A control 22 bp rRNA representing the V3 region of E. coli 16s was also synthesized in vitro.

First hybridization: The sample was combined with two 20 bp DNA probe molecules (5′-AACGTCAATGAGCAAAGGTATT-3′, SEQ ID NO. 9; and 5′-CTGAAAGTACTTTACAACCCG-3′, SEQ ID NO. 10) designed to bind to the 3′ and 5′ rRNA regions flanking the 22 bp target sequence (having the same sequence as the 22 bp control rRNA). In particular, a 4 μL aliquot of the 90 bp sample rRNA (1 μM) was mixed with 2 μL of the two DNA probe molecules (1 μM each) and 4 μL of water to bind to the flanking sequences of the target 22 bp rRNA. The mixture was heated at 95° C. for 3 minutes, then left on the bench for 15 minutes.

First enzymatic digestion: 4 μL of buffer (comprising 50 mM Tris-HCl (pH 8.3), 75 mM KCl, 3 mM MgCl₂, and 10 mM DTT) and 2 μL of 5 U/μL RNase H were added to the mixture resulting from the first hybridization reaction to digest the regions of the RNA bound to the DNA probes (producing 22 bp rRNA fragments). The enzyme/rRNA mixture was incubated at 37° C. for 20 minutes, then 2 μL of 10 mM EDTA (pH 8) was added to deactivate the enzyme.

Second hybridization: The mixture resulting from the first enzymatic digestion was combined with a different DNA probe molecule (5′-AACTTTACTCCCTTCCTCCCGGCCCCCCCCCCCCCCC-3; SEQ ID NO. 11) targeted to the V3 region of E. coli 16s (having the same sequence as the control 22 bp rRNA). For hybridization, 4 μL of 1 μM probe molecule and 5 μL of SSC buffer were added, and the resulting mixture heated at 95° C. for 3 minutes, then left on the bench for 15 minutes.

Second enzymatic digestion: 4 μL of 5 U/μL RNase 1f was added to the mixture resulting from the second hybridization reaction. The enzyme/rRNA mixture was incubated at 37° C. for 20 minutes, then 5 μL of 100 mM HgCl₂ added to deactivate the enzyme.

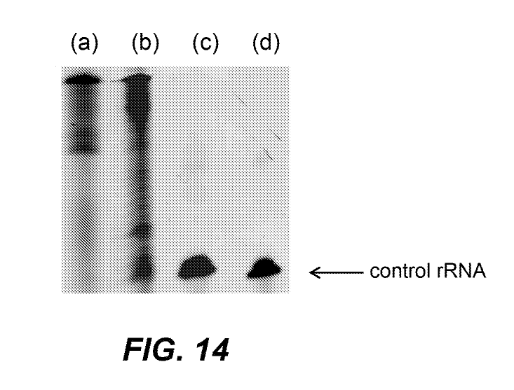

Gel electrophoresis was run for (a) 90 bp rRNA after the first hybridization reaction (b) 90 bp rRNA after the first hybridization reaction and first enzymatic digestion with RNase H, (c) 90 bp rRNA after the first hybridization reaction, first enzymatic digestion with RNase H, second hybridization reaction, and second enzymatic digestion with RNase 1f, and (d) control 22 bp rRNA treated only to the second hybridization as described above. A 5 μL aliquot of each of (a)-(d) was mixed with 6 μL of 2× high density loading dye (Invitrogen) and heated for 70° C. for 3 minutes, then 10 μL was run on a 15% TBE-urea gel. The gel was run at 180 v for 2 hours, and stained with GelRed™ 20 minutes before visualization. Results of the electrophoresis are shown in FIG. 14. The results indicate that the first pair of DNA probe molecules successfully direct RNAse H to the appropriate areas of the 90 bp rRNA to cut out the 22 bp target of interest, and the second probe molecule then binds the 22 bp target and protects it from degradation by RNAse A/T1.

### Example 4

The effect of using multiple probe molecules targeted to different regions of a microbial RNA molecule was investigated. Whole RNA (wRNA) was isolated from whole E. coli through phenol chloroform extraction.

For hybridization, the sample wRNA was combined with a set of six probe molecules, each targeted towards a different region of E. coli 16s rRNA that displays a sequence divergence of at least 4 bp among bacterial species (E. coli, Salmonella, Staph., and Candida). The probe molecules were:

#1)
(SEQ ID NO. 12)
5′-ATGGCAAGAGGCCCGAAGGTCCCCCCCCCCCCCCCCC-3′
#2)
(SEQ ID NO. 13)
5′-CCTCCATCAGGCAGTTTCCCAGCCCCCCCCCCCCCCC-3′
#3)
(SEQ ID NO. 14)
5′-TCAGACCAGCTAGGGATCGTCGCCCCCCCCCCCCCCC-3′
#4)
(SEQ ID NO. 15)
5′-AACTTTACTCCCTTCCTCCCCGCCCCCCCCCCCCCCC-3′
#5)
(SEQ ID NO. 16)
5′-TCAGTCTTCGTCCAGGGGGCCGCCCCCCCCCCCCCCC-3′
#6)
(SEQ ID NO. 17)
5′-GCCATGCAGCACCTGTCTCACGCCCCCCCCCCCCCCC-3′

A 4 μL aliquot of 1 mg/mL sample wRNA was mixed with 2 μL of a 10 μM mixture of the 6 probe molecules or 10 μM of probe #1 (SEQ ID NO. 12), 5 μL of SSC buffer, and 3 μL of water. The resulting mixture was heated at 95° C. for 3 minutes, then left on the bench for 15 minutes.

For digestion, 2 μL of buffer (comprising 100 mM NaCl, 50 mM Tris-HCl (pH 7.9), 10 mM MgCl₂, and 1 mM DTT) and 4 μL of a mixture of RNase A and RNase T1 (comprising 40 units of RNase A and 20 units of RNase T1) were added to the hybridization reaction mixture. The enzyme/wRNA mixture was incubated at 37° C. for 20 minutes, then 5 μL of 100 mM HgCl₂ was added to deactivate the enzymes.

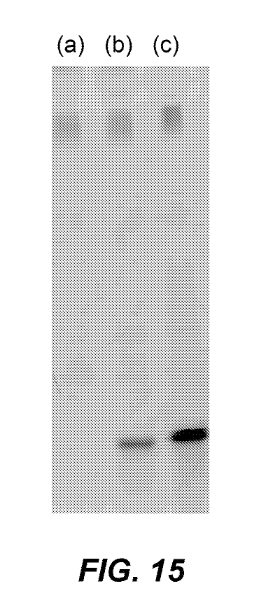

Gel electrophoresis was run for (a) E. coli wRNA combined with the RNase A/T1 mixture, (b) E. coli wRNA combined one probe molecule, with enzyme treatment, and (c) E. coli wRNA combined with all 6 probe molecules, with enzyme treatment. A 5 μL aliquot of each of (a)-(d) was mixed with 6 μL of 2× high density loading dye (Invitrogen) and heated for 70° C. for 3 minutes, then 10 μL was run on a 15% TBE-urea gel. The gel was run at 180 v for 2 hours, and stained with GelRed™ 20 minutes before visualization. Results are shown in FIG. 15. The results indicate that the RNAse mixture degraded all RNA unless the probe molecules were added to bind to and protect the 22 bp sequence of interest, and that more probe molecules lead to more target/probe complexes, isolating more 22 bp targets. The combination of six probe molecules showed a nearly 6-fold increase in target signature patterns as compared to a single probe molecule.

### Example 5

Multiple probe molecules were used in an assay to investigate the effect on the frequency of signal (signature patterns) detected for a sample of whole rRNA. Specifically, a sample of rRNA was isolated from E. coli (16s and 23s) through sucrose density gradient centrifugation.

For hybridization, the sample rRNA was combined with the 6 probe molecules of Example 4, targeted towards a different region of E. coli 16s rRNA. For each probe molecule, 4 μL of 1 mg/mL sample rRNA was mixed with 2 μL of 1 μM probe molecule, 5 μL of saline-sodium citrate (SSC) buffer, and 3 μL of water. Each mixture was heated at 95° C. for 3 minutes, then left on the bench for 15 minutes.

For digestion, two different RNase A/T1 mixtures were used: a first mixture comprising 30 units of RNase A and 150 units of RNase T1, and a second mixture comprising 20 units of RNase A and 50 units of RNase T1. For each, 2 μL of buffer (comprising 100 mM NaCl, 50 mM Tris-HCl (pH 7.9), 10 mM MgCl₂, and 1 mM dithiothreitol (DTT)) and 4 μL of the RNase A/T1 mixture was added to separate hybridization reactions as described above. Each enzyme/rRNA mixtures were incubated at 37° C. for 20 minutes, then 5 μL of 100 mM HgCl₂ was added to deactivate the enzymes.

The entire samples were then analyzed in separate nanopore experiments (separate nanopore systems). The samples were added to the cis side of α-hemolysin nanopore system as described in Example 2. Current was recorded for 30 minutes, and the data analyzed to identify 3-level signature patterns corresponding to the target/probe complexes resulting from the 6 probe molecules. The total number of signals (signature patterns as described in connection to FIG. 2) over the 30-minute period was normalized to the single-channel time recorded (number of nanopores×the amount of time analyzed).

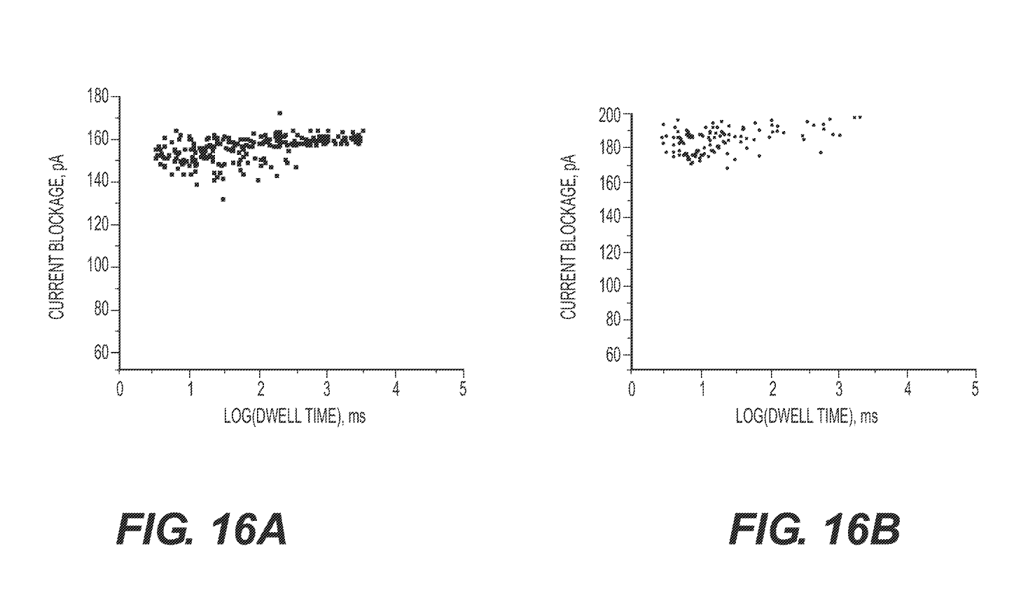

FIGS. 16A and 16B show a scatter plot of the length of the current blocks (ms) vs. the magnitude of the current blockage (pA) for all signature patterns. In FIG. 16A (sample prepared with the first enzymatic mixture), 234 signals were recorded for 38.5 single-channel minutes (SCM), where SCM refers to the number of signals divided by the number of nanopores of the system and the number of minutes recorded. Thus, 6 signature patterns were observed per SCM (i.e., 6 signature patterns per nanopore per minute). In FIG. 16B (sample prepared with the second enzymatic mixture), 98 signals were recorded for 9.75 SCM, corresponding to 10 signature patterns per nanopore per minute. While the assay of FIG. 16A produced more total signals, more SCMs were recorded. Thus, FIG. 16B indicates a more effective assay.

### Example 6

Multiple probe molecules were used in an assay to investigate the effect of a set of probe molecules vs. a single probe molecule on the frequency of signal (signature patterns) detected for a sample of whole RNA (wRNA). A sample of RNA was isolated from E. coli through phenol chloroform extraction.

Two different hybridization reactions were performed. In the first reaction, sample wRNA was combined with probe molecule #1 of Example 4. In the second reaction, sample wRNA was combined with all 6 probe molecules of Example 4. For each probe molecule, 4 μL of 1 mg/mL sample wRNA was mixed with 2 μL of 1 μM probe molecule(s), 5 μL of saline-sodium citrate (SSC) buffer, and 3 μL of water. Each mixture was heated at 95° C. for 3 minutes, then left on the bench for 15 minutes.

For digestion, 2 μL of buffer (comprising 100 mM NaCl, 50 mM Tris-HCl (pH 7.9), 10 mM MgCl₂, and 1 mM dithiothreitol (DTT)) and 4 μL of a mixture of RNase A and RNase T1 (comprising 30 units of RNase A and 150 units of RNase T1) were added to each mixture resulting from the hybridization reactions. The enzyme/wRNA mixtures were incubated at 37° C. for 20 minutes, then 5 μL of 100 mM HgCl₂ was added to deactivate the enzymes.

The two sample wRNAs following digestion with the enzymatic mixture were added to the cis side of α-hemolysin nanopore system prepared according to Example 2. Current was recorded for 30 minutes, and the data analyzed to identify 3-level signature patterns corresponding to the target/probe complexes resulting from the probe molecule(s). The total number of signals (signature patterns) over the 30-minute period was normalized to the single-channel time recorded (number of nanopores×the amount of time analyzed).

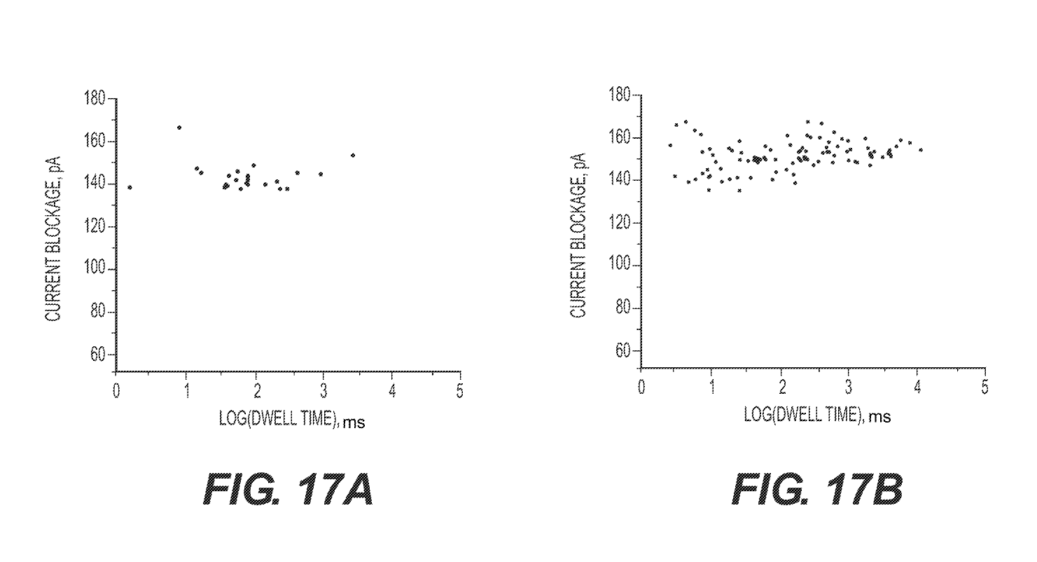

FIGS. 17A and 17B show the length of the current blocks (ms) vs. the magnitude of the current blockage (pA). In FIG. 17A (sample prepared hybridized to probe molecule #1, only), 24 signals were recorded for 58 SCM, corresponding to 0.41 signature patterns per nanopore per minute. For the set of 6 probe molecules, shown in FIG. 17B, 97 signals were recorded for 35 SCM, corresponding to 2.8 signature patterns per nanopore per minute. Thus, using multiple probe molecules in preparation of the wRNA sample resulted in a higher signal frequency as compared to a single probe molecule, demonstrating an increase in sensitivity can be achieved by targeting multiple regions of one rRNA target.

It is intended that the specification and examples be considered as exemplary only, with a true scope and spirit of the present disclosure being indicated by the following claims.

1. A method of detecting a target nucleic acid in a sample, the method comprising:
combining the sample with at least one probe molecule having a sequence fully complementary or partially complementary to the target nucleic acid, the target nucleic acid being single-stranded, such that the probe molecule hybridizes to the target nucleic acid;
combining the sample with one or more enzymes to produce a probe/target complex;
applying a voltage across a nanopore system while the probe/target complex is on a first side of a partition of the nanopore system, the partition including a nanopore defining a channel; and
analyzing an electrical current of the nanopore system over time, wherein a presence of the target nucleic acid in the sample is indicated by a signature current pattern comprising a level or a series of levels with magnitudes of current and durations respectively different from magnitudes of current and durations of levels of each of an electrical current that occurs with the sample in absence of the at least one probe molecule and an electrical current that occurs with the at least one probe molecule in absence of the target nucleic acid.

2. The method of claim 1, wherein the sample comprises a parent nucleic acid that includes the sequence of the target nucleic acid, such that the target of the probe/target complex is a fragment of the parent nucleic acid.

3. The method of claim 2, wherein combining the sample with the one or more enzymes cleaves the parent nucleic acid to isolate the probe/target complex from a remainder of the parent nucleic acid.

4. (canceled)

5. The method of claim 1, wherein the sample comprises non-target single-stranded nucleic acids, and the one or more enzymes digest the non-target single-stranded nucleic acids.

6. The method of claim 1, wherein the one or more enzymes comprises RNase A, RNase 1, RNase 1f, RNase P, RNase PhyM, RNase T1, RNase T2, RNase U2, or a mixture thereof.

7. (canceled)

8. The method of claim 2, wherein the target nucleic acid comprises from 18 to 25 nucleotides in length, and the parent nucleic acid comprises more than 30 nucleotides in length.

9. (canceled)

10. The method of claim 2, wherein the at least one probe molecule is a third DNA probe molecule, the method further comprising combining the sample with a first DNA probe molecule having a sequence complementary to a sequence of the parent nucleic acid flanking a 3′ end of the target nucleic acid and a second DNA probe molecule complementary to a sequence of the parent nucleic acid flanking a 5′ end of the target nucleic acid, before combining the sample with the third DNA probe molecule.

11. The method of claim 10, further comprising combining the sample with one or more enzymes after combining the sample with the first and second DNA probe molecules and before combining the sample with the third DNA probe molecule.

12. The method of claim 11, wherein the one or more enzymes combined with the sample after the first and second DNA probe molecules and before the third DNA probe molecule cleaves the parent nucleic acid at the 3′ end and the 5′ end of the target nucleic acid to release the target nucleic acid from the parent nucleic acid.

13. The method of claim 11, wherein the one or more enzymes combined with the sample after the first and second DNA probe molecules and before the third DNA probe molecule comprises RNase H.

14. (canceled)

15. The method of claim 1, wherein the signature current pattern comprises three consecutive levels of electrical current, each level having a magnitude of current different from the other levels of the three consecutive levels.

16. The method of claim 1, wherein the signature current pattern corresponds to:
(a) trapping the target/probe complex in a trans opening of the nanopore; or
(b) detaching the target nucleic acid from the at least one probe molecule of the target/probe complex and translocating at least one of the probe molecule or the target nucleic acid completely through the nanopore, from a cis opening to a trans opening of the nanopore, or from the trans opening to the cis opening of the nanopore.

17. The method of claim 1, wherein the sample further comprises non-target nucleic acids, and the signature current pattern distinguishes the target nucleic acid from the non-target nucleic acids.

18. The method of claim 1, wherein the target nucleic acid is a first target nucleic acid, and the sample further comprises a second target nucleic acid, wherein a sequence of the second target nucleic acid differs from the sequence of the first nucleic acid by 1 or 2 nucleotides, and wherein the signature current pattern distinguishes the presence of the first nucleic acid in the sample from a presence of the second nucleic acid in the sample.

19. The method of claim 1, wherein the nanopore system comprises a first chamber that includes the first side of the partition and a second chamber that includes a second side of the partition, and the first chamber is in contact with a negative electrode of the nanopore system.

20. The method of claim 1, wherein the nanopore system comprises a first chamber that includes the first side of the partition and a second chamber that includes a second side of the partition, and the second chamber is in contact with a negative electrode of the nanopore system.

21. The method of claim 1, wherein the nanopore comprises a biological nanopore or a synthetic nanopore, and wherein the channel has a minimum cross-sectional size ranging from about 1.2 nm to about 1.8 nm.

22. (canceled)

23. (canceled)

24. The method of claim 1, wherein the channel is defined by a surface of the nanopore that includes a plurality of molecules or chemical functional groups facing radially inward.

25. (canceled)

26. (canceled)

27. The method of claim 1, wherein the nanopore comprises Staphylococcus aureus α-hemolysin, Mycobacterium smegmatis porin A, or Escherichia coli CsgG.

28. The method of claim 1, wherein the partition of the nanopore system comprises a plurality of nanopores chosen from biological nanopores, synthetic nanopores, or a combination thereof.

29. The method of claim 1, wherein the target nucleic acid comprises a fragment of whole RNA or a microRNA.

30. The method of claim 1, wherein the target nucleic acid comprises a fragment of microbial rRNA.

31. The method of claim 2, further comprising quantifying an amount of the target nucleic acid in the sample, quantifying an amount of the parent target nucleic acid in the sample, or quantifying the amount of the target nucleic acid and the amount of the parent target nucleic acid in the sample.

32. (canceled)

33. The method of claim 1, wherein the target nucleic acid is a biomarker of at least one of a genetic disease, an environmental disease, an organism genotype, a pathogen, a resistance to an antiobiotic, or a bacterial infection.

34-43. (canceled)

44. A method of detecting a target nucleic acid in a sample, the method comprising:
combining the sample with a first probe molecule and a second probe molecule, wherein:
the sample comprises a parent nucleic acid that includes a sequence of the target nucleic acid;
the first probe molecule has a sequence complementary to a sequence of the parent nucleic acid flanking a 3′ end of the target nucleic acid; and
the second probe molecule has a sequence complementary to a sequence of the parent nucleic acid flanking a 5′ end of the target nucleic acid;
adding at least one first enzyme to the sample;
combining the sample with a third probe molecule having a sequence fully complementary or partially complementary to the sequence of the target nucleic acid, such that the third probe molecule hybridizes to the target nucleic acid;
adding at least one second enzyme to the sample to produce a target/probe complex; and
detecting the target/probe complex with a nanopore system.

45. The method of claim 44, wherein the at least one first enzyme comprises RNase H; and the at least one second enzyme is chosen from RNase A, RNase 1, RNase 1f, RNase P, RNase PhyM, RNase T1, RNase T2, RNase U2, or a mixture thereof.

46. The method of claim 44, wherein detecting the target/probe complex comprises:
applying a voltage across a nanopore system while the probe/target complex is on a first side of a partition of the nanopore system, the partition including a nanopore defining a channel; and
analyzing an electrical current of the nanopore system over time, wherein a presence of the target nucleic acid in the sample is indicated by a signature current pattern comprising a level or a series of levels with magnitudes of current and durations respectively different from magnitudes of current and durations of levels of each of an electrical current that occurs with the sample in absence of the third probe molecule and an electrical current that occurs with the third probe molecule in absence of the target nucleic acid.
